(19) United States
(12) Patent Application Publication
(10) Pub. No.: US 2025/0046404 A1
(43) Pub. Date: Feb. 6, 2025

(54) SYSTEMS AND METHODS RELATING TO A MACHINE LEARNING PREDICTION MODEL FOR PREDICTING MOLECULAR TARGETS OF SMALL MOLECULES

(71) Applicants: Diego Galeano, Marambure Luque (PY); Afshin Beheshti, Medford, MA (US); Bianca Gonzales Cerqueira, Olmos Park, TX (US); Justin Reese, Milledgville, GA (US)

(72) Inventors: Diego Galeano, Marambure Luque (PY); Afshin Beheshti, Medford, MA (US); Bianca Gonzales Cerqueira, Olmos Park, TX (US); Justin Reese, Milledgville, GA (US)

(73) Assignee: KBR WYLE SERVICES, LLC, Houston, TX (US)

(21) Appl. No.: 18/792,311

(22) Filed: Aug. 1, 2024

Related U.S. Application Data
(60) Provisional application No. 63/517,572, filed on Aug. 3, 2023.

(51) Int. Cl. G16C 20/70; G16C 20/30

(52) U.S. Cl. CPC G16C 20/70; G16C 20/30

(57) ABSTRACT

A computer implemented method may collect a set of known drug compounds from a database, and create augmented datasets with labeled and unlabeled chemical data and biological data. A computer implemented method may train a neural network using the expanded training set to generate a prediction score of bioactivity of candidate drug compounds towards the biological target, wherein the contribution of the second plurality of unlabeled samples in training the neural network is scaled by a parameter. A computer implemented method may output one or more refined neural network models capable of generating candidate drug compounds with predicted activity. A computer implemented method may generate a candidate drug compound by inputting a candidate drug compound with chemical data into the trained neural network model.

### INCORPORATION BY REFERENCE TO ANY PRIORITY APPLICATIONS

This application claims the benefit of U.S. Provisional Application No. 63/517,572, filed Aug. 3, 2023, which is hereby incorporated by reference.

### BACKGROUND

### Field

This disclosure relates to predicting chemical bioactivity of small molecules. More particularly, this disclosure relates to predicting biological activity of small molecule drugs towards DNA, RNA and protein targets where the existing biological data for a set of biological targets is sparse.

### Description of the Related Art

Current efforts are ongoing to computationally predict drug molecules for chemical bioactivity. Several of these initiatives include Quantitative Structure-Activity Relationship (QSAR) models, deep learning models, molecular docking models, and generative models, all of which fall under the category of machine learning. All of these models work with large compound libraries to screen for potential drug candidates.

QSAR models analyze the relationship between the chemical structure of molecules and their biological activity. Specific machine learning algorithms, such as linear regression, random forest, and support vector machines, are trained on known chemical structures and corresponding bioactivity data to predict the activity of new molecules. Deep learning models aim to extract complex features from molecular structures and learn intricate patterns in large datasets. Techniques like graph convolutional networks (GCNs) and recurrent neural networks (RNNs) have been applied to predict bioactivity based on molecular graphs or sequential data. Molecular docking models are used to predict the binding affinity between a small molecule and a target protein. Machine learning approaches can enhance docking accuracy by incorporating additional features, such as target-based descriptors, into the docking process.

The objective of generative models, such as generative adversarial networks (GANs) and variational autoencoders (VAEs), is to produce novel drug-like molecules. By training on large chemical databases, GANs attempt to understand the influence of molecular structure on bioactivity and generate novel compounds as candidates. These models are useful for chemical space exploration and the discovery of novel drug candidates; however these models can still be limited to producing only drug candidates that are similar or nearly identical to the original training set. Another limitation is that these models often produce compounds that cannot be synthesized.

Disease models of interest may only have small datasets, which presents significant challenges for machine learning, including but not limited to insufficient representation, overfitting, substantial variance, and restricted coverage of feature space. Small datasets typically lack the diversity and quantity of examples used to effectively capture the problem's complexity, resulting in ineffective generalization and unreliable predictions. The models trained on small datasets can overfit, memorizing the limited data and exhibiting poor performance on unseen examples. High variance and limited feature space coverage impede the models' ability to recognize true patterns and make the models extremely sensitive to small changes in parameters.

### SUMMARY

An aspect of the present disclosure is directed to methods and systems that predict chemical bioactivity of small molecules. For example, some embodiments are directed to targeting miRNAs with small molecules, which have been implicated in human diseases from cancers to COVID-19. The present disclosure provides for a generalized deep learning framework for predicting biological activity of small molecules on miRNA targets on the basis of the small molecule's chemical structure and the miRNA targets' sequence information. A novel objective function that allows the neural network to learn chemical space from a large body of chemical structures is used to overcome the limitation of sparse information about biological activity of small molecules on miRNAs.

An aspect of the present disclosure is directed to expanding the size of training sets where limited bioactive chemical datasets are available. Embodiments according to the present disclosure include improved neural networks trained with expanded datasets, where the improved neural networks were validated by experimentation. The present disclosure provides for a loss function that scales the contribution of any unlabeled data sets towards training of a neural network. Another aspect of the present disclosure is directed to a framework to predict bioactive small molecules that are chemically dissimilar from those available in training. An aspect is directed to generating novel compounds, or existing but unused compounds, for activity against biological targets in model organisms.

For purposes of summarizing the present disclosure and the advantages achieved over the any related work, certain objects and advantages of the present disclosure have been described herein. Of course, it is to be understood that not necessarily all such objects or advantages may be achieved in accordance with any particular embodiment of the present disclosure. Thus, for example, those skilled in the art will recognize that the present disclosure may be embodied or carried out in a manner that achieves or optimizes one advantage or group of advantages as taught herein without necessarily achieving other objects or advantages as may be taught or suggested herein.

In some examples, the techniques described herein relate to a computer-implemented method for automatically training an artificial intelligence engine to generate candidate drug compounds, wherein the computer-implemented method includes: collecting a set of known drug compounds from a database; creating a first training set including chemical data and biological data associated with the set of known drug compounds; wherein the chemical data includes structural information of the drug compounds and the biological data includes bioactivity information of the drug compounds towards a biological target; creating a second training set including chemical data associated with drug compounds with unknown biological activity towards a biological target; combining the first training set and the second training set to form an expanded training set; and training a neural network using the expanded training set to generate a prediction score of bioactivity of candidate drug compounds towards the biological target; wherein the contribution of the second plurality of unlabeled samples in training the neural network is scaled by a parameter α; wherein α is greater than zero and less than one; outputting one or more refined neural network models capable of generating candidate drug compounds with predicted activity; generating a candidate drug compound by inputting a candidate drug compound with chemical data into the trained neural network model, and assessing the candidate drug compound predicted activity using the trained neural network model.

In some examples, the techniques described herein relate to a computer-implemented method for increasing the size of training set data for an artificial intelligence engine, wherein the computer-implemented method includes: providing a first training set including a first plurality of training samples, wherein each training sample includes associated input data labels and corresponding output labels; providing a second training set including a second plurality of partially-labeled samples, wherein the partially labeled samples include associated input data labels without corresponding data output labels; wherein the second training set is larger than the first training set; optionally applying a data augmentation technique to the initial training set to generate augmented training samples, wherein the data augmentation technique introduces variations in the input data while preserving the corresponding output labels; combining the first training set and the second training set to form an expanded training set; and training a neural network using the expanded training set to generate a prediction score of bioactivity of candidate drug compounds; wherein the contribution of the second plurality of unlabeled samples in training a neural network is scaled by a parameter α; wherein α is greater than zero.

In some examples, the techniques described herein relate to a computer-implemented method for training a neural network to engine to generate candidate drug compounds affecting one or more biological targets, wherein the computer-implemented method includes: collecting a set of known drug compounds from a database; creating a first training set including chemical data and biological data associated with the set of known drug compounds; wherein the chemical data includes structural information of the drug compounds and the biological data includes bioactivity information of the drug compounds towards one or more biological target; wherein each biological target has associated sequence information; creating a second training set including chemical data associated with drug compounds with unknown biological activity towards one or more biological targets; combining the first training set and the second training set to form an expanded training set; calculating a sequence similarity score between biological targets based on the sequence information of the biological targets; training a neural network using the expanded training set to generate a prediction score of bioactivity of candidate drug compounds towards a biological target; wherein the contribution of each biological target towards other biological targets is weighted by the sequence similarity score; wherein the contribution of the second plurality of unlabeled samples is reduced as compared to the first plurality of labeled data in training the neural network; and outputting one or more refined neural network models capable of generating candidate drug compounds with predicted activity.

All of these embodiments are intended to be within the scope of the present disclosure herein disclosed. These and other embodiments of the present disclosure will become readily apparent to those skilled in the art from the following detailed description of the preferred embodiments, the present disclosure not being limited to any particular preferred embodiment(s) disclosed.

### BRIEF DESCRIPTION OF THE DRAWINGS

The features and advantages of the methods and compositions described herein will become more fully apparent from the following description and appended claims, taken in conjunction with the accompanying drawings. These drawings depict only several embodiments in accordance with the present disclosure and are not to be considered limiting of their scope. In the drawings, similar reference numbers or symbols typically identify similar components, unless context dictates otherwise. In some instances, the drawings may not be drawn to scale.

FIG. 1A shows an example machine learning system for analyzing chemical and biological data.

FIG. 1B shows an example machine learning method for analyzing chemical and biological data.

FIG. 2A depicts a first training set and a second training set.

FIG. 2B illustrates how the first training set and the second training set may be combined to form an expanded training set.

FIG. 3 illustrates an expanded training set that may be used to train a neural network model.

FIG. 4A and FIG. 4B show the performance of a neural network trained using methods and systems described herein compared to other machine learning models.

FIG. 5A and FIG. 5B show the performance of a neural network trained using methods and systems described herein compared to other machine learning models.

FIG. 6 shows a plot illustrating an average predicted ranking of known bioactive small molecules.

FIGS. 7A and 7B illustrate the prediction performance at retrieving active small molecules against fifty randomly selected miRNA targets in Homo sapiens from a pool of 4,000 small molecules in a leave-one-out procedure.

FIGS. 8A, 8B, and 8C are heatmaps showing an enriched Mode of Action (MoA) and indications of small molecules predicted by a neural network trained using the methods and systems described herein (sChemNET) to target miRNAs in Homo sapiens.

FIGS. 9A and 9B illustrate an example of a trained neural network for predicting small molecules that bind to target miRNAs in other species.

FIG. 10A illustrates the performance of a machine learning model in predicting the bioactivity of small molecules in Mus musculus (mice)

FIG. 10B illustrates the performance of a machine learning model in predicting the bioactivity of small molecules in Rattus norvegicus (rats).

### DETAILED DESCRIPTION

In the following detailed description, reference is made to the accompanying drawings, which form a part thereof. In the drawings, similar symbols typically identify similar components, unless context dictates otherwise. Thus, in some embodiments, part numbers may be used for similar components in multiple figures, or part numbers may vary depending on figure to figure. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and made part of this disclosure.

MicroRNAs (miRNAs) are a class of non-coding RNAs that play a role in post-transcriptional gene regulation by modulating the levels of transcripts. Dysregulation of miRNAs is associated with various metabolic and cardiovascular disorders, cancers, hepatitis, and infectious diseases such as COVID-19. MicroRNAs can be highly stable and circulate in the blood of diseased individuals, providing biomarkers of disease and potential therapeutic targets.

Oligonucleotide inhibitors, having a complementary structure to characterized miRNAs associated with particular diseases, are being developed to inhibit these miRNA targets. For instance, oligonucleotides complementary to miR-122 are being developed to treat Hepatitis C virus, and an antisense oligonucleotide to miR-2392 is being explored for treating COVID-19. Similarly, synthetic miRNAs are under development for the treatment of cancers to regulate certain oncogenes and block tumor growth. For example, mimics of miR-34 have been designed for the treatment of various human cancers. However, the use of oligonucleotides to target miRNAs or mimic miRNA activity has proven difficult due to high toxicity of the oligonucleotides and challenges associated with delivery of the oligonucleotides.

Alternatively, miRNAs may be targeted with small molecules. However, information about the bioactivity of small molecules with target miRNAs is limited. Presently, the bioactivity of 131 small molecules has been assessed against 126 human miRNAs. For a majority of the 126 miRNAs, less than 10 bioactive, small molecules have been identified. To put this in perspective, the human genome includes over 2,600 mature miRNAs, and there are many more small molecules that exist or that may be synthesized to target each of the human miRNAs. The lack of information about small molecules and their effect on miRNAs presents a technical problem for predicting the bioactivity of new, unassessed small molecules with one or more miRNA targets. Previous models for predicting miRNA targets of small molecules have limited applicability and could only predict the bioactivity of the 131 known small molecules on the 126 known miRNA targets, which had been already experimentally identified.

The present application is directed, in part, to systems, methods, and computer readable media to predict the bioactivity of known and unknown small molecules with miRNA targets. A novel deep learning framework is described herein and generates the probabilities that a given small molecule can modulate a given miRNA target based on its chemical features with increased accuracy and efficiency. In contrast to existing methods, the systems and methods described herein can be applied to identify novel bioactive chemicals from any chemical library. For example, novel small molecules may be identified based on information about the 2D chemical structure of the compounds. The systems and methods described herein can be applied across species to identify small molecules for targeting disease in human patients.

The present disclosure provides for models with the ability to integrate chemical structure information for small molecules with unknown effect on miRNAs. Surprisingly, even training on a small known dataset, the disclosed models achieve good prediction performance for the bioactivities of a wide set of small molecules. The present disclosure further provides for supplementing smaller chemical datasets currently available for model organisms by applying a cross-species data integration and by integrating miRNAs sequence information.

### Systems for Predicting Molecular Targets of Small Molecules in Small Bioactive Chemical Datasets

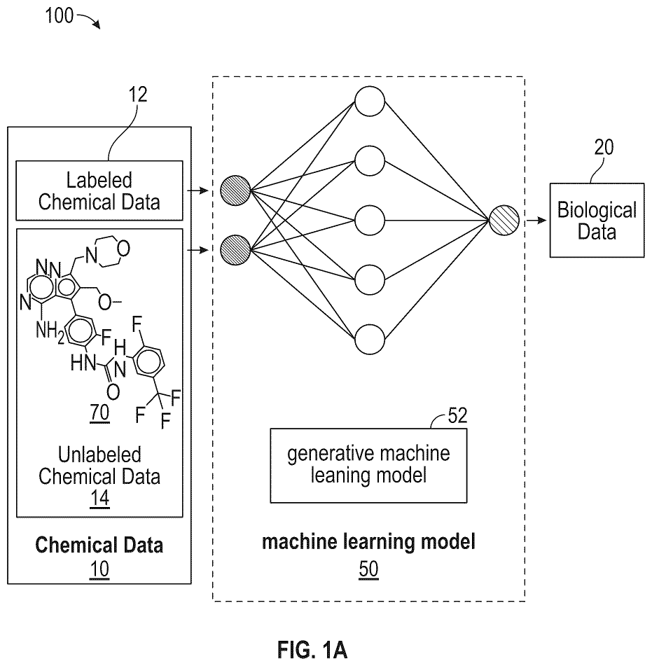

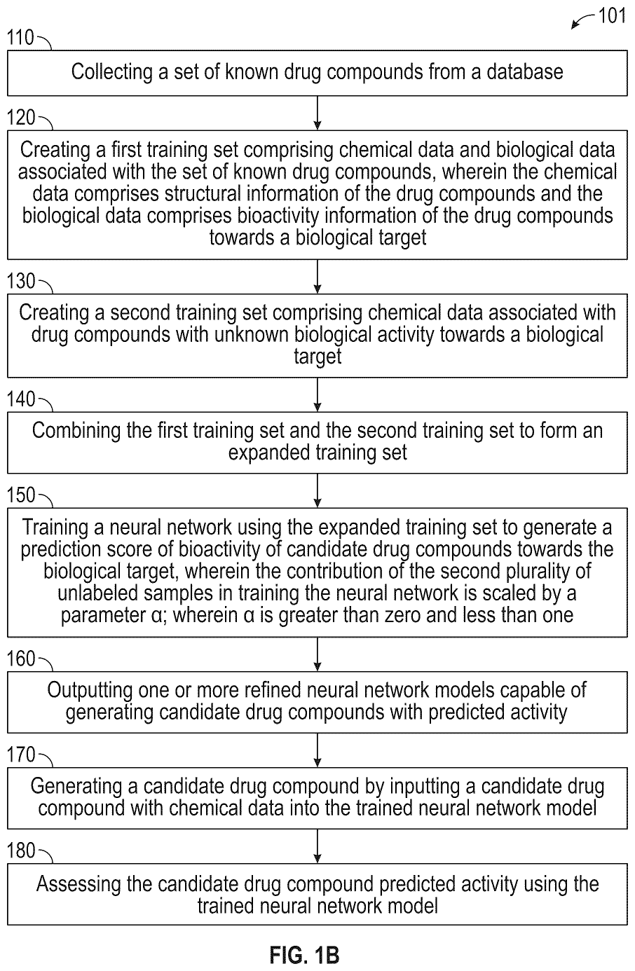

The present disclosure relates to novel machine learning systems designed to predict the bioactivities of a wide set of small molecules. FIG. 1A shows an example machine learning system 100 for analyzing chemical data 10 and biological data 20. The chemical data 10 may include labeled chemical data 12. The chemical data 10 may include unlabeled chemical data 14. The chemical data 10 may include both the labeled chemical data 12 and the unlabeled chemical data 14. In examples, the labeled chemical data 12 includes structural information of a small molecule and is labeled with paired biological data 20 for the small molecule. For example, a first small molecule may have been clinically observed to have bioactivity to a first biological target, no biological activity towards a second biological target and unknown biological activity towards a third biological target. Unlabeled chemical data 14 may include structural information of a small molecule. In examples, chemical data 14 may lack paired biological data for the small molecule. The machine learning system 100 may apply a machine learning model 150 to utilize advanced machine learning algorithms to analyze the chemical data 10 and biological data 20.

The machine learning system 100 may apply a machine learning model 150 to train on chemical data 10 and biological data 20. The trained machine learning model 151 may be applied to a novel candidate small molecule 70. In examples, the trained machine learning model 151 may predict the bioactivity of the small molecule 70. The molecule shown in FIG. 1A is one example of a candidate small molecule, and the specific compound is 1-(4-(4-amino-6-(methoxymethyl)-7-(morpholinomethyl)pyrrolo[2,1-f][1,2,4]triazin-5-yl)-2-fluorophenyl)-3-(2-fluoro-5-(trifluoromethyl)phenyl)urea. This non-limiting example was determined to have a biological activity towards an miRNA target while other examples included in the unlabeled data set were not predicted to have biological activity.

In examples, the machine learning system 100 may predict a novel candidate small molecule 70 by applying a machine leaning model 150 to train on labeled chemical data 12 and unlabeled chemical data 14. In examples, the novel candidate small molecule 70 may be included in the unlabeled chemical data 14. In examples, the trained model 151 (not shown) may provide an updated prediction for the previously unlabeled candidate small molecule 170. Note however, that machine learning models typically do not train on the exact data they will be used to predict for in order to keep clear separation between training and testing data. In examples, the machine learning system 100 may also generate a novel candidate small molecule 70 by applying a generative machine learning model 152 (shown as an embodiment of 150) to train on chemical data 10 and biological data 20. A generative machine learning model 152 may learn the underlying structure of molecules and may generate new compounds 170 with desired bioactivity profiles. Generative machine learning models 152 may include the same architecture as shown in FIG. 1A, but may also have a different architecture with or without a neural network architecture.

By way of example, the present disclosure provides for a deep-learning predictive model that incorporates information about small molecules with known and as-yet-unknown biological activity on miRNAs in a neural network model to predict small molecules targeting miRNAs (or their downstream targets) on the basis of their chemical structure alone. For the unlabeled chemical data 14 approximately 2,400 “unlabeled” small molecules were used with a smaller number of “labeled” small molecules 12. The labeled chemical data 12 was a set of small molecules known to affect miRNA expression level, directly or indirectly. The machine learning model 50 in this example included a two-layered neural network where the chemical structure information of the labeled and unlabeled small molecules is fed into the model and distributed over a set of hidden layers of nodes. The output layer of the network represents each of the miRNAs and the model outputs a predicted score for each miRNA based on a given small molecule's chemical feature. Accordingly, the trained machine learning model generates a score mapping between the chemical features of a small molecule and a set of miRNAs.

In some embodiments, a machine learning system may include a small molecule-miRNA interaction predictor that includes a multi-task two-layered feed-forward neural network. In examples, a machine learning system may include a set of input chemical features. In examples, the machine learning system may include a set of hidden units fully connected to the input features (with a dropout parameter p, batch normalization, and Relu activation function). In examples, the machine learning system may include a set of hidden units fully connected to the input features that may in term be connected to a set of output units.

Output units may represent each of the miRNAs. The output units may be fully connected to the hidden units (with a dropout parameter p and sigmoid activation function). The model may be trained on the input features and the output units by minimizing an appropriate loss function with a corresponding optimizer.

Consistent with the disclosure and the disclosed examples, the following hyperparameters may be simultaneously or independently optimized: number of hidden units (n), unlabelled regularization parameter (α), number of epochs (e), learning rate (lr), and dropout (p). In some embodiments, a Bayesian optimization procedure may be used for hyperparameter search with the following bounds e∈[100, 300], lr∈[0.0001, 0.1], p∈[0.1, 0.5], α∈[0.001,0.3]. The number of hidden units may be tested for a discrete set of n∈{8,16,32}.

When predicting the biological activity of small molecules, one or more loss functions may be employed to train the model and improve the accuracy of the predictions. For regression tasks, the Mean Squared Error (MSE) is commonly used, which measures the average squared difference between the predicted and true activity values. Another option is the Mean Absolute Error (MAE), which calculates the average absolute difference. For binary classification tasks (active or inactive), Binary Cross-Entropy is often employed to evaluate the dissimilarity between predicted probabilities and true labels. In the case of multiclass classification, Categorical Cross-Entropy is frequently utilized, encouraging the model to assign high probabilities to the correct class. Additionally, ranking losses such as pairwise ranking loss or listwise loss can be employed when the objective is to rank molecules based on their activity levels. As described above the choice of a specific loss function depends on the nature of the problem and the desired behavior of the model; however, a preferred loss function, which may include elements from the loss function described above, will integrate chemical structure information of small molecules as-yet-unknown to affect miRNAs directly or their miRNA-regulated transcriptional programs. A specific implementation is described in more detail below. In brief, the loss function provides an approach that may be applied to infer novel bioactive chemicals from various chemical libraries with information about the 2D chemical structure of the compounds.

In some embodiments, a machine learning model may be trained and tested using small-size chemical datasets with labeled information about the bioactivities of each small molecule on miRNAs. The Small Molecule to miRNA (SM2miR) database may be used to obtain curated information on small molecules known to affect the expression levels of either specific miRNAs or of their corresponding mRNA targets. For each targeted miRNA, the number of bioactive small molecules in a data set may vary, for example between 5 to 35, and its distribution may follow a long-tailed pattern. In examples, other data sets may be used that may include more or fewer entries with different distributions. A wide range of 2-D structural information may be obtained for unlabeled drug compounds from the Drug Repositioning Hub, which contains a large set of structurally and therapeutically diverse small molecules that have reached clinical drug development, including most FDA-approved drugs. 6,302 unique unlabeled small molecules may be used together with 131 small molecules from the SM2miR database to build a unique set of 6,433 small molecules. Each small molecule may then be represented by a 167-dimensional chemical feature vector based on their MACCS chemical fingerprints. In examples, sequence similarities between miRNAs may be obtained by calculating the Needleman-Wunsch score using their mature sequences from the miRBase database.

The use of a large amount of chemical information by sChemNET can simulate a realistic scenario in which a small molecule that is biologically active against a miRNA may be recovered from a large pool of chemicals. To this end, for each known bioactive small molecule-miRNA association, a test set containing a large number of small molecules may be constructed wherein only a few or even one may be experimentally determined as being bioactive and the remaining ones may be randomly selected small molecules as yet unknown to affect the targeted miRNA. For example, for each known bioactive small molecule-miRNA association, a test set containing 4,000 small molecules may be constructed, where only one was experimentally determined to be bioactive, and 3,999 can be randomly selected small molecules as yet unknown to affect the targeted miRNA. In examples, high performance of the model can be observed when assessed based on the percentage of known bioactive small molecules that could be retrieved amongst the top predicted small molecules.

While the specific examples described herein offer one amongst many possible approaches to solving a machine learning problem, alternative methods can be employed at various steps of the process. Consistent with the present disclosure, a diverse range of algorithms, techniques, and frameworks, may be used to, for example, predict new compounds, integrate sequence information, expand the size of the training set, and improve the performance of machine learning models by efficient handling of unlabeled data sets. The machine learning models described herein include specific steps that that are tailored to the structure and dimensionality of the features that machine learning models take as input data or generate as output predictions/classifications. For example, the size, shape, and type of input and output data for a machine learning model can vary depending on the specific problem being addressed. Accordingly, depending on the nature of the problem, alternative machine learning methods may be substituted for certain steps.

The size of the input data may refer to the number of observations available for the model to learn from. In examples, the biological data may include sequence information that may be condensed and represented by a sequence similarity coefficient. In other examples, the sequence information may directly include a polynucleotide sequence for a biological target, thereby allowing for potential patterns to be extracted from connections between drugs and the sequence.

In examples, a sequence similarity coefficient and polynucleotide sequence have different structure and dimensionality, and consistent with the present disclosure, may affect the architecture and design of the machine learning model. The type of input data may vary widely, ranging from numerical values to categorical data (e.g., labels, text), or even more complex forms like sequences or graphs. In classification problems, the output may be categorical labels indicating the class membership of each input sample. For regression tasks, the output is typically numerical values representing predictions or estimates. Other types of tasks may have different outputs, such as generating sequences, ranking small molecules for likelihood of affecting a biological target, or suggesting a chemical to test in a “design of experiments” format.

### Machine Learning Model Types

Methods and systems according to the present disclosure may employ machine learning models 150 from one or more categories. For example, a machine learning model 50 may be a neural network system that introduces a multi-layered architecture, comprising interconnected nodes, which mimic the processing and learning mechanisms. The system may incorporate of deep learning methodologies, enabling the network to automatically learn hierarchical representations of data features. By iteratively processing and transforming the input data through multiple layers, the neural network system may capture meaningful patterns. The neural network system may incorporate advanced techniques such as regularization, dropout, and batch normalization to mitigate overfitting, enhance generalization capabilities, and improve the system's robustness in handling unseen data. Embodiments of the present disclosure include deep learning models, which are a subset of neural networks that comprise multiple layers of interconnected artificial neurons. Furthermore, the present disclosure provides for incorporating loss functions, described further herein, and activation functions, such as rectified linear units (ReLU), softmax, and sigmoid, to introduce non-linearities and enhance the system's ability to model complex data distributions accurately.

Machine learning models 150 may leverage both supervised and unsupervised learning algorithms to adaptively adjust weights to fit the training data. Generally, supervised learning models are trained using labeled datasets, where the input data is paired with corresponding target labels. These models learn to map input features to desired outputs, allowing them to make predictions when applied to new, unseen data. Examples include linear regression, logistic regression, decision trees, random forests, support vector machines (SVMs), and neural networks.

Unsupervised learning models are trained on unlabeled data, meaning these models lack predefined target labels. These models aim to discover hidden patterns, structures, or relationships in data. Clustering algorithms, such as k-means, hierarchical clustering, and Gaussian mixture models, may be used to group similar data points representing either chemical or biological features together. In other examples, dimensionality reduction techniques like principal component analysis (PCA) and t-SNE may extract essential features and/or reduce the complexity of the data.

In some embodiments, semi-supervised learning models may use a combination of labeled and unlabeled data for training the machine learning model. These models may leverage the limited labeled data to guide the learning process while leveraging unlabeled data to improve generalization and accuracy. Machine learning models according to the present disclosure may also leverage knowledge learned from one task or domain to improve performance on a different but related task or domain. Such models may transfer pre-trained knowledge from a first dataset to a second dataset allowing better generalization with limited data.

An example of the present disclosure is directed to expanding the size of training sets and handling of the unlabeled data to supplement an initial small training set. Accordingly, machine learning models according to the present disclosure may be open ended and combine with other machine learning methods to make a final prediction. By leveraging the diversity and collective wisdom of multiple models, ensemble methods enhance accuracy, reduce overfitting, and improve generalization.

A two-layered fully connected neural network model, also known as a shallow neural network, may include an input layer, a hidden layer, and an output layer. The two-layered fully connected neural network model is considered shallow (i.e., it has a small number of layers compared to deeper architectures, but may still capture basic patterns and relationships in data. Each layer may be composed of interconnected artificial neurons or nodes, and the connections between the neurons will have associated weights. The input layer is responsible for accepting the input data or features. Each neuron in the input layer represents a feature, and the number of neurons in this layer corresponds to the dimensionality of the input data. The values of the input neurons may be directly determined by the input features.

In terms of the model architecture, the hidden layer may be positioned between the input and output layers and may be responsible for processing and transforming the input data. Each neuron in the hidden layer may be configured to receive inputs from all the neurons in the previous layer (in this case, the input layer) and applies an activation function to produce an output value. The activation function may introduce non-linearity to the model, enabling the model to learn complex patterns in the data. The number of neurons in the hidden layer may be determined by the complexity of the problem and the desired capacity of the model.

The output layer may be configured to receive inputs from the neurons in the hidden layer and produces the final output of the neural network. The number of neurons in the output layer depends on the nature of the problem. For example, in a binary classification task, there would typically be one neuron in the output layer, representing the probability or prediction of one class. In a multi-class classification task, the number of neurons may correspond to the number of classes, and the outputs may be represented as probabilities (using, for example, an activation function like softmax).

Each neuron in the hidden and output layers may be connected to every neuron in the previous layer (fully connected). These connections have associated weights, which represent the strength or importance of the connection. During training, the model may be configured to adjust these weights iteratively using optimization algorithms, such as gradient descent, to minimize the difference between predicted outputs and the true labels. The weight values may be learned through backpropagation, where the error may be propagated backward from the output layer to the hidden layer, updating the weights to improve the model's performance.

Various modifications and enhancements can be made to the model described herein. For example, the model may be modified by one or more of incorporating regularization techniques, choosing different activation functions, and/or using different optimization algorithms, to improve its performance and generalization abilities.

### Types of Chemical Data

The chemical data 10 may include labeled chemical data 12, and unlabeled chemical data 14. Chemical data 10 may encompass molecular structures, represented through, for example, atom connectivity graphs or Simplified Molecular Input Line Entry System (SMILES) strings, as well as descriptors that quantify the properties of compounds, including molecular weight, partition coefficients, and topological indices. Chemical fingerprints may encode binary patterns to represent the presence or absence of specific substructures and physicochemical properties such as solubility, and boiling point. Additionally, some data such as toxicity data and binding coefficients may be included as chemical data 10.

The size, shape, and type of input data for a machine learning model can vary depending on the specific problem being addressed. The shape of the input data represents the structure and dimensionality of the features and factors into determining the architecture and design of a given model. In general, machine learning models take input data, often referred to as features, and generate output predictions or classifications. The size of the input data refers to the number of observations or samples available for the model to learn from. For example, if the task is to classify small molecules, the size of the input data would correspond to the total number of molecules in the dataset and the number of features provided for each molecule. The type of input data can vary, ranging from numerical values to categorical data (e.g., labels and text).

Chemical data may be represented using various descriptors, such as MACCs (Molecular ACCess System) keys. MACCs are a set of binary fingerprints that encode the presence or absence of specific chemical substructures or features in a molecule. While the number of binary fingerprints may vary, in some embodiments a MACCs key may include 166 fingerprints, or 127 fingerprints. Each MACC key corresponds to a specific chemical pattern, such as the presence of a particular functional group, a ring system, or a specific atom arrangement. The MACCs descriptor system provides a compact representation of chemical structures, allowing for efficient analysis of molecules. Each MACC key can be considered as a bit in a binary fingerprint, with “1” indicating the presence of the corresponding chemical feature and “0” representing its absence.

In examples, the process and system as described may be configured to provide predictions for biological activity for small molecules that may be chemically dissimilar from the molecules in the training set. There are several methods for calculating chemical similarity. For example, the Tanimoto index is a measure of similarity between two sets. In the context of chemical informatics, the Tanimoto index is commonly used to quantify the similarity between two chemical compounds or fingerprints. The Tanimoto index may be calculated by dividing the intersection of the two sets by the union of the sets. Mathematically, it can be expressed in the equation below as:

$$Tanimoto ( A , B ) = ( A ⋂ B ) / ( \vert A \vert + \vert B \vert - A ⋂ B )$$

A and B represent two sets, and |A| and |B| denote the sizes (cardinalities) of sets A and B, respectively. When applied to chemical compounds, the sets A and B typically represent the presence or absence of specific chemical features, often encoded as binary fingerprints or descriptors. The Tanimoto index quantifies the degree of overlap between the chemical features present in the two compounds. The resulting Tanimoto index value ranges between 0 and 1, with 0 indicating no similarity (no common features) and 1 indicating complete similarity (identical features). Accordingly, a higher Tanimoto index suggests a greater degree of similarity between the chemical compounds. MACCs may also be used for similarity searching. MACCs fingerprints also enable the identification of molecules that possess specific chemical features of interest, and by comparing MACCs fingerprints, researchers can identify molecules that share similar chemical substructures.

The following chemical datasets are non-limiting examples of suitable chemical data 10. In some embodiments, labeled chemical data 12 may include the SM2miR database 20 version Apr. 27, 2015. This example database includes manually curated associations between small molecules and miRNAs. Each small molecule in the labeled chemical data 12 may be mapped to a corresponding PubChem identifier (CID). Additionally, the corresponding miRNA may be mapped to a miRbase identifier. Through the SM2miR database, a total of 4,244 small molecule-miRNA associations were extracted across 18 different species.

In an example, only miRNAs with a minimum of five small molecule associations may be retained for each organism under investigation. For Homo sapiens, 1,102 associations involving 131 small molecules and 126 miRNA targets may be utilized. In the case of Mus musculus, 272 associations may be obtained, involving 44 small molecules and 43 miRNAs. Rattus norvergicus contributed 78 associations, comprising 32 small molecules and 13 miRNAs.

In examples, the Drug Repositioning Hub may be used to expand the chemical library of small molecules with no known activity against miRNAs. This hub contains a diverse collection of small molecules that have undergone clinical trials for various indications. Specifically, “non-replicated” small molecules may be selected, ensuring they possess different chemical structures than those available in the SM2miR database. Consequently, the final set of small molecules for Homo sapiens, Mus musculus, and Rattus norvergicus amounted to 6,302, 6,281, and 6,294, respectively.

### Types of Biological Data

The labeled chemical data 12 includes structural information of a small molecule and may be labeled with paired biological data 20 for the small molecule. The size and type of the biological data 20, here used as output data, will depend on the specific task at hand. In classification problems, the output may be categorical labels indicating the class membership of each input sample, for example, biologically active or not biologically active. For regression tasks, the output may be numerical values representing predictions. Other types of tasks may have different output requirements, such as generating sequences, ranking items, or detecting anomalies.

Specific biological data 20 may include, but is not limited to, gene expression data, protein sequences and/or 3D structures. This type of information may contribute to understanding protein function, interactions, and drug binding. In some embodiments, some biological information may be used as labels for the chemical data 10, and are not strictly used as output data. For example, protein sequences and three-dimensionality of biological structures contribute to understanding protein function, interactions, and drug binding. Accordingly, the protein sequences of biological targets may be incorporated into an input layer of the model, such as by labelling that a chemical may be active towards a protein, and including the protein sequence.

In some embodiments, biological data such as miRNA sequence similarity may be incorporated as a weight in the loss function. Accordingly, in some embodiments chemical and biological data may be integrated without being expressly included into an input or output layer. The miRNA mature sequences, for the biological targets, may be obtained from an miRBase database using miRNA identifiers. miRNA sequence similarities may be computed using mature miRNA sequences using global alignment in BioPython v1.76. Sequence similarity scores may be then normalized between 0 and 1.

### Training

In examples, methods for training a machine learning model 50 are described. For example, machine learning model 50 may be constructed by preparing a representative and diverse dataset, with the types described above. This dataset includes input features and corresponding target labels, and to ensure reliable model training, the dataset may be divided into training, validation, and testing subsets, allowing for proper evaluation of the trained model's performance on unseen data.

Prior to training, feature engineering techniques may be employed to optimize the input features. These techniques encompass normalization, scaling, encoding categorical variables, handling missing values, and dimensionality reduction. By performing feature engineering, the input features may suitably transformed and prepared for optimal model performance, enabling the model to learn from the most relevant information in the data. In some preferred embodiments, the relevant information includes the chemical data 112 in MACCs key format, and biological data in a binary category label for active/inactive.

The present disclosure provides for selecting the appropriate model architecture tailored to the specific problem and data characteristics. As described above, various model types may be chosen, such as neural networks, decision trees, support vector machines, or ensemble methods, each with its own strengths and suitability for different tasks. The model architecture determines the arrangement of the model's layers, nodes, and connections, impacting the model's ability to learn relationships in the data. In some embodiments, the model architecture may include a set of input chemical features, a set of hidden units fully connected to the input features (with a dropout parameter p, batch normalization, and Relu activation function), followed by a set of output units, representing each of the miRNAs, fully connected to the hidden units (with a dropout parameter p and sigmoid activation function).

In some embodiments, the system may initiate the training process by initializing model parameters, such as weights and biases. Proper initialization sets a starting point for the model to learn and refine its parameters based on the provided input data. This initialization may be performed using techniques like random initialization or pre-training with related datasets. In some embodiments, the unlabeled chemical data 14 may be initialized with an initial value of zero. In some embodiments, the initial value of zero may be allowed for float. In some embodiments, depending on the availability of known negative results, the value of zero may be fixed and required to be a zero corresponding to no biological activity.

During the training phase, a training algorithm, such as gradient descent, stochastic gradient descent, or evolutionary algorithms, may be employed. An algorithm iteratively updates the model's parameters to minimize a loss function, which quantifies the discrepancy between the model's predictions and the actual target labels. Optimization techniques, such as learning rate schedules, adaptive learning rates, or momentum, may be employed to enhance convergence and avoid getting trapped in local optima.

When predicting the biological activity of small molecules, one or more loss functions can be employed to assess the accuracy of the predictions. For regression tasks, the Mean Squared Error (MSE) may be commonly used, measuring the average squared difference between the predicted and true activity values. Another option is the Mean Absolute Error (MAE), which calculates the average absolute difference. For binary classification tasks (active or inactive), Binary Cross-Entropy is often employed to evaluate the dissimilarity between predicted probabilities and true labels. In the case of multiclass classification, Categorical Cross-Entropy is frequently utilized, encouraging the model to assign high probabilities to the correct class. Additionally, ranking losses such as pairwise ranking loss or listwise loss can be employed when the objective is to rank molecules based on their activity levels. In some embodiments, the loss function may be minimized using an ADAM optimizer with default parameters in Tensorflow/Keras v2.8.0 (beta1=0.9, beta2=0.999, epsilon=1e-7).

Described further herein, the present disclosure provides for specific loss functions that may integrate chemical and biological information. For example, a loss function may include a first summation term that applies a fitting constraint to the labeled chemical information. This first term may allow the machine learning model 50 to learn a high prediction score for known associations between small molecules and miRNAs.

An example loss function may also include labeled information available for all other miRNAs, such that the relative learning contribution for each biological target on unknown small molecules may be weighted based on sequence similarities of the miRNA target to other targets.

Another term in the loss function may include a hyperparameter to control the relative importance of unlabeled small molecules that may be assigned low prediction scores towards each targeted miRNA during learning/initialization.

Hyperparameter tuning may be a sensitive step in the training process. Hyperparameters, such as learning rate, regularization parameters, or network architecture configurations, may be fine-tuned to optimize the model's performance. Techniques like grid search, random search, or Bayesian optimization can be employed to systematically explore a hyperparameter space and identify the optimal configuration for the model.

Evaluation and validation also play a role in the training process. Periodically, a model's performance may be assessed on a separate validation dataset. Metrics such as loss, accuracy, or other relevant performance indicators may be monitored to evaluate the model's progress. Based on this evaluation, adjustments to the model architecture or hyperparameters may be made to enhance its performance and generalization capabilities.

To prevent overfitting and determine the ideal stopping point, stopping criteria may be defined. These criteria can be based on the convergence of the loss function or early stopping strategies that leverage the model's performance on the validation dataset. By employing appropriate stopping criteria, the training process may be effectively managed, ensuring the model may be trained to the point where it maximizes its predictive capabilities without overfitting the training data.

### Predicting Activity of Small Molecules

One embodiment may be directed to determining the activity of small molecules to known biological targets. Most FDA-approved drugs are known to exert their therapeutic effect by binding to specific proteins in the cell. Of all the human proteins, only an estimated 700 have been successfully drugged to date. Accordingly, the present disclosure provides for an in silico approach that can predict miRNA targets targeted by of small molecules on the basis of the small molecule chemical structure alone.

A model, referred to herein as sChemNET, is a non-limiting example prepared consistent with the present disclosure. sChemNET may be trained with labeled and unlabeled small molecules and the trained model may be used to rank 4,000 small molecules in a test set by a predicted activity scores. sChemNET may obtain good performance with n=16, α=0.286, dropout=0.174, and learning rate=0.0346. sChemNET architecture may include one or more hyperparameters—number of hidden units (n), unlabeled regularization parameter (a), number of epochs, learning rate, and dropout. A Bayesian optimization approach was employed for hyperparameter search based on a LOOCV of the small molecules known to target miR-224-5p; a miRNA that was randomly selected and excluded from further evaluation analysis.

The performance of sChemNET was assessed based on the percentage of known bioactive small molecules that could be retrieved amongst the top 100, 300, 500, or 1000 predicted small molecules. The performance of sChemNET, with and without (sᵤᵥ=1) integrating sequence similarity information, may be compared with other machine learning methods that were trained using the same input feature information as sChemNET: XGBoost, Logistic Regression (LR), Random Forest (RF), and a Feed-Forward Neural Network (FNN), and two other approaches that rank each of the 4,000 small molecules in the test set based on: (i) the maximum Tanimoto chemical similarity to the set of bioactive small molecules in the training set (chemical similarity) or (ii) random scores assigned to each small molecule when sampling from a uniform distribution between 0 and 1 (random). sChemNET outperformed the compared methods at different numbers of predictions retrieved by 1-9% for the top 100 of small molecules retrieved from the test set, 7-21% (top 300), 5-33% (top 500), and 8-29% in the top 1000. sChemNET achieves good prediction performance even without using sequence similarity information in the loss function but with a small reduction in prediction performance of about 1.81-3.62% across the different top-K thresholds.

In some embodiments, the model provides mechanisms to interpret and explain the reasoning behind its predictions, allowing users to understand the factors influencing the outcomes and enhancing transparency. In other embodiments, the model may only predict whether a small molecule might affect a miRNA, but not provide information about the molecular mechanism of action of the small molecule.

### Method for Predicting Molecular Targets of Small Molecules in Small Bioactive Chemical Datasets

FIG. 1B is an example computer-implemented process for predicting the binding probability of a small molecule with a target miRNA. In some embodiments, the process 101 may include more or fewer steps. For example, one or more of the steps of process 101 may be performed in a different order or simultaneously with respect to one or more of the other steps of process 101. In some embodiments, different machine learning methods may include more or fewer steps. For example, a classification model may include different steps, or experimental design than a regression model in order to generate a predicted biological activity of a novel small molecule.

The process 101 may begin at step 110 where one or more processors of a computing system may retrieve a set of small molecules from a database. The small molecules in the database may be known to be bioactive against at least one miRNA target. Small molecules known to be bioactive against at least one miRNA may be referred to herein as “labeled” small molecules. In some embodiments, the database may store chemical properties associated with each of the small molecules known to be bioactive against at least one miRNA target. The chemical properties may include a chemical fingerprint of each known small molecule. For example, the chemical fingerprint may be a 2-D chemical fingerprint, such as a MACCS fingerprint, a PubChem fingerprint, a custom fingerprint scheme, or the like.

In some embodiments, the one or more processors may receive a user input that includes a set of small molecules. In some embodiments, the one or more processors may receive a chemical formula or a structural formula for the small molecule(s). The one or more processors may determine the chemical fingerprint for the small molecule(s) based on the chemical formula and/or the structural formula.

In some embodiments, the one or more processors may retrieve a known bioactivity for at least one of the small molecules against at least one target miRNA. Bioactive small molecules may inhibit or promote the expression of the miRNA, inhibit or promote the activity of the target miRNA, etc. In some embodiments, the bioactivity of a small molecule against an miRNA may be given a bioactivity score. The bioactivity score may be selected from a range of values, e.g. from 0 to 1 with 1 representing the highest bioactivity and 0 representing no bioactivity. The bioactivity score may be selected from a category of values, e.g. from 0 or 1 with 1 representing bioactivity and 0 representing no bioactivity. For example, the bioactivity of a first small molecule known to affect a first miRNA target may be stored in the database, and the one or more processors may retrieve the bioactivity of the first small molecule. In some embodiments, the one or more processors, receive the bioactivity of the small molecule(s) via a user input.

The process 101 may move to step 120 where the one or more processors may create a first training set. The first training set may be referred to herein as a “labeled small molecule” training set. The one or more processors may create the first training set using the information and data collected in step 110. The one or more processors may compile chemical data and biological data associated with the set of small molecules with known bioactivity against the target miRNA(s). Chemical data may include chemical properties associated with the small molecules, such as a chemical fingerprint, a molecular similarity score, etc. The biological data may include bioactivity information of the small molecules a biological target, such as a miRNA target.

The first training set may include at least one feature vector associated with at least one small molecule with a known bioactivity against at least one target miRNA. The feature vector may include the chemical properties and/or the biological properties associated with the small molecule(s). The feature vector may include a chemical fingerprint of the at least one small molecule. The chemical fingerprint may be converted to an “N”-D chemical feature vector. The chemical feature vector may include a plurality of binary values indicating the presence or absence of chemical structures or motifs. The length of the chemical feature vector may be based on the chemical fingerprint employed. For example, the chemical feature vector may be at least a 166-dimensional chemical feature vector in embodiments where a MACCS fingerprint is used.

In some embodiments, the feature vector may include additional properties associated with the small molecule such as an miRNA sequence similarity score. In some embodiments, the miRNA sequence similarity score may be determined by calculating a Needleman-Wunsch score. The Needleman-Wunsch score for the small molecule and particular miRNA target may be calculated based on the mature sequence of the target miRNA. For example, a chemical feature vector may be a 167-dimensional chemical feature vector. The 167-dimensional chemical feature vector may include the 166 MACCS keys appended with the Needleman-Wunsch score. In some embodiments, the sequence similarity score may be stored in the database and may be retrieved in step 110. In some embodiments, the one or more processors may retrieve the mature form of the miRNA target from the miRbase database and calculate the sequence similarity score for the small molecule.

The training set may include the bioactivity score of the small molecule(s) known to be bioactive against the at least one miRNAs. The bioactivity scores may be stored separately from the feature vectors as “true” values that may be used to train a model.

The bioactivity may be stored in a binary vector of the length of the total number of miRNAs (e.g. N=126 for Homo sapiens data). In some examples, a vector has the following format=[1, 0, 0, 0, 1, . . . ].

The process 101 may move to step 130 where the one or more processors may create a second training set. The second training set may be referred to herein as an “unlabeled small molecule” training set. The second training set may include chemical data and/or biological data associated with at least one small molecule with unknown biological activity towards at least one target miRNA. Similar to the first training set, the one or more processors may retrieve a chemical data associated with the small molecule(s) with unknown bioactivity toward the target miRNA(s). For example, the database may store chemical data associated with the small molecules with unknown bioactivity, which may include chemical formulas, structural formulas, chemical fingerprints such as a MACCS fingerprint, etc. In some embodiments, the data associated with the small molecule(s) with unknown bioactivity toward the target miRNA(s) may be stored in a separate database from the data associated with the small molecule(s) known to be bioactive towards the target miRNA(s).

Using the chemical data, the one or more processors may create a chemical feature vector for each of the small molecules with unknown bioactivity toward the target miRNA(s) as described above. For example, the one or more processors may retrieve the MACCS fingerprint for each small molecule and create a 167-dimensional feature vector using the MACCS fingerprint and appending a calculated sequence similarity score, e.g. a Needleman-Wunsch score. In some embodiments, the one or more processors may set the bioactivity of the small molecules with unknown bioactivity to a negligible value, such as zero.

In some embodiments, the processor may move to step 140, where the one or more processors may combine the first training set and the second training set to form an expanded training set. The expanded training set may include at least one feature vector from the first training set and a plurality of feature vectors from the second training set. In some embodiments, the expanded training set may include feature vectors from the first training set that may be known to be bioactive against the target miRNA(s). For example, a feature vector associated with a first small molecule known to target a first miRNA may be include in the expanded training set. In another example, a first feature vector associated with a first small molecule known to target a first miRNA and a second feature vector associated with a second small molecule known to target a second miRNA may be included in the expanded training set.

In some examples, the distribution of known “bioactive” small molecules varies per miRNA (e.g. from 5 to 35 in Homo sapiens data), therefore, the number of labelled small molecule will vary for each miRNA. In some examples, this number does not change in the expanded training set, because a possible goal of the expanded training set is to increase the number of small molecules without adding new information about labels.

The feature vectors of small molecules with unknown bioactivity from the second training set may be added to the feature vectors from the first training set to create the expanded training set.

In some examples, data may be supplemented by a row-wise concatenation and/or a column-wise concatenation. In some example, the rows have small molecules and in the column have features, but the choice of labels for the rows and columns may be arbitrary. Row-wise concatenation and column-wise concatenation are operations used in data manipulation and matrix handling. Row-wise concatenation involves combining two or more data sets or matrices by stacking them vertically, for example, placing one dataset or matrix below another. This operation increases the number of rows in the resulting dataset or matrix while maintaining the original number of columns. On the other hand, column-wise concatenation involves merging datasets or matrices horizontally, for example, placing one dataset or matrix beside another. These types of operations increase the number of columns in the resulting dataset or matrix while keeping the original number of rows intact.

In some embodiments, the feature vectors from the second training set may be chosen at random. we select a subset of the small molecule at random, together with their corresponding feature vectors.

For a given miRNA u, we select at random the unlabelled small molecules (yet-unknown to affect miRNA u). These are over 2,400 small molecules to be kept as part of the training set, the remaining we use it for prediction (or in the LOOCV procedure). The use of random selection is to discourage the model to learn the specifics of the mapping chemical feature->label, when the label=0. Rather, we want the model to learn about what makes chemical feature->label, when label=1 (bioactive)—that is, to learn meaningful chemical features from the bioactive ones but also using the unlabeled to better learn to discriminate the bioactive ones in chemical space.

In some embodiments, the number of feature vectors from the second training set may be proportional to the number of feature vectors from the first training set. For example, the number of feature vectors from the second training set may be at least 5 times greater, 10 times greater, at least 15 times greater, at least 20 times greater, at least 25 times greater, at least 30 times greater, at least 35 times greater, at least 40 times greater, at least 45 times greater, at least 50 times greater, or any other suitable multiple greater than the number of feature vectors from the first training set.

In some embodiments, the number of feature vectors from the second training set may represent a proportion of the total number of feature vectors in the second training set. For example, about 5%, about 10%, about 15%, about 20%, about 25%, about 30%, about 33.33% or ⅓, about 35%, about 40%, about 45%, about 50%, or any other suitable percentage of the feature vectors in the second training set may be included. The remaining portion of the second training set that is not included in the expanded training set may be used as a testing set. In some embodiments, a feature vector associated with a small molecule with unknown bioactivity may be analyzed using the methods described herein and may be left out of the expanded training set.

The process 101 may move to step 150 where the one or more processors may train a model using the expanded training set to generate a predicted bioactivity score of candidate small molecules towards at least one target miRNA. At step 150, a computer implemented method may include the step of training a neural network using the expanded training set to generate a prediction score of bioactivity of candidate drug compounds towards the biological target, wherein the contribution of the second plurality of unlabeled samples in training the neural network is scaled by a parameter α; wherein α is greater than zero and less than one. The model may be a neural network comprised of a plurality of layers. The neural network may include an input layer, at least one hidden layer, and an output layer. In some embodiments, the neural network may include at least two hidden layers, at least three hidden layers, at least four hidden layers, or at least 5 hidden layers.

The input layer may include binary inputs corresponding with each value in a small molecule feature vector. The at least one hidden layer may include a plurality of nodes. In some embodiments, the at least one hidden layer may include at least 5 nodes, at least 10 nodes, at least 15 nodes, at least 20 nodes, at least 25 nodes, at least 35 nodes, at least 40 nodes. The number of nodes in the at least one hidden layer may be optimized over time based on training and subsequent model performance. The output layer may include a predicted bioactivity value for each target miRNA. The predicted bioactivity value may represent a percent chance that a small molecule will affect the expression levels of a target miRNA. In some embodiments, the predicted bioactivity value may represent a percent chance that a small molecule will bind to a target miRNA thereby affecting the expression levels of the target miRNA or its mRNA targets.

In some examples, and throughout the disclosure, a predicted score and an output probability may be used interchangeably as two different ways of representing a model's prediction for a given input. A predicted score may be a real-valued number that represents the model's estimated value for a specific outcome. The score could be continuous or discrete, depending on the type of problem being solved. For example, in regression tasks, the predicted score might represent a numeric value, such as predicting the price of a house, or a binding constant for a miRNA target. In binary classification tasks, the predicted score may represent the model's confidence in assigning an instance to a particular class (e.g., 0 for one class and 1 for the other), or any value in between.

Output probability may be a value between 0 and 1, representing the likelihood or confidence that a given input belongs to a particular class in a classification problem. In binary classification, the output probability represents the probability of the positive class (class 1) and, by extension, 1 minus that probability represents the probability of the negative class (class 0). For example, an output probability of 0.8 for the positive class means the model is 80% confident that the input belongs to that class.

One distinction, if applicable, is that a predicted score is often used in regression tasks or in binary classification tasks when the model is not probabilistic, while output probabilities are commonly used in probabilistic models like logistic regression, softmax regression, or deep learning models with softmax activation in the output layer.

In some embodiments, the model may provide a ranking of small molecules based on the predicted bioactivity value for each small molecule.

Each layer may include a plurality of nodes or neurons that may be each connected to at least one neuron in each adjacent layer. In some embodiments, the neural network may be fully connected, and each node may be connected to every node in each adjacent layer. For example, each node in the input layer may be connected to every node in the at least one hidden layer, and each node in the at least one hidden layer may be connected to each node in the output layer.

The neural network may include one or more hyperparameters. The neural network hyperparameters may include a number of nodes, a number of epochs, a learning rate, a dropout rate, and/or at least one loss function hyperparameters. In some embodiments, the one or more hyperparameters may be optimized using a set of small molecules known to bind to a particular target miRNA. The target miRNA used to optimize the hyperparameters may be selected based on a target miRNA and/or candidate drug compound to be assessed by the neural network after training. For example, if a group of candidate drug compounds are to be assessed against a first target miRNA, a second target miRNA may be used to optimize the hyperparameter(s).

In some embodiments, a Bayesian optimization approach may be used to identify desirable or optimal values for the one or more hyperparameters. For example, a group of small molecules known to bind with miR-224-5p may be input into a neural network, and using a leave-one-out cross validation (LOOCV) and a Bayesian optimization approach, values for the one or more hyperparameters may be identified. In this embodiment, the number of nodes in each hidden layer was identified as 16; the learning rate was identified as 0.0346, and the dropout rate was identified as 0.174. The values for the one or more loss function hyperparameters may be also identified as discussed above.

The neural network may be trained using a loss function. In some embodiments, the loss function may be a mean squared error function. The loss function may weigh the predicted bioactivity of small molecules with known bioactivity and small molecules with unknown bioactivity separately. In some embodiments, the loss function may include a first weighted summation for labeled small molecules with known bioactivity and a second weighted summation for unlabeled small molecules with unknown bioactivity.

As discussed above, the loss function may include at least one loss function hyperparameter. The loss function hyperparameters may include one or more of a sequence similarity weighting parameter and/or an unknown bioactivity weighting parameter. The sequence similarity weighting parameter may be represented as the term “s.” The sequence similarity weighting parameter may use the sequence similarity between small molecule with known bioactivity toward at least target miRNA to weigh the difference between a predicted bioactivity and a true bioactivity of small molecules known to be bioactive against the at least one target miRNA. The unknown bioactivity weighting parameter may be represented as the term alpha, or “a.” The unknown bioactivity weighting parameter may weigh the predicted bioactivity for small compounds with unknown bioactivity.

In some embodiments, the loss function may be defined by the equation below:

$$L = \frac{1}{2} {\sum }_{( i , v ) is labeled} {{s}_{u v} ( {\hat{y}}_{i v} - {y}_{i v} )}^{2} + \frac{α}{2} {\sum }_{( i , v ) is unlabeled} {\hat{y}}_{i v}^{2}$$

“i” may represent the ith small molecule, and “u” may represent a target miRNA. “ŷᵢᵥ” may represent a bioactivity of the ith small molecule with a chemical feature “xᵢ” against the uth target miRNA that is predicted by the neural network. “yᵢᵥ” may represent a known bioactivity of the ith small molecule with a chemical feature, “xᵢ,” against the target miRNA “u.” “ŷᵢᵥ” and “yᵢᵥ” may be values within a defined range. For example, “ŷᵢᵥ,” and “yᵢᵥ” may be values between 0 and 1 with 0 representing no likelihood of bioactivity and 1 indicating that a small molecule is bioactive against the target miRNA. In some embodiments, “yᵢᵥ” for small molecules with a known bioactivity against a target miRNA may be set to 1 during training. In some embodiments, the “yᵢᵥ” for small molecules with an unknown bioactivity against a target miRNA may be set to 0 during training. In some embodiments, the “yᵢᵥ” for small molecules with an unknown bioactivity against a target miRNA may initially be set to 0 during training and allowed to float across epochs.

As discussed above, the term “s” in the equation above may represent the sequence similarity weighting parameter, and the term “a” in the equation above may represent the unknown bioactivity weighting parameter. “s” may be set based on a sequence similarity score for the ith small molecule. In some embodiments, “s” may be equal to the sequence similarity score, such as a Needleman-Wunsch score, retrieved and/or calculated for the ith small molecule in steps 110, 120, and/or 130. In some embodiments, a may be a value that is greater than or equal to zero but less than or equal to two. In some embodiments, a may be a value that is greater than or equal to zero but less than 1.

In some embodiments, s and/or a may be optimized using the Bayesian optimization approach described above. Returning the example above, a group of small molecules known to bind with miR-224-5p may be input into a neural network, and using a leave-one-out cross validation (LOOCV) and Bayesian optimization approach, optimal values for the loss function hyperparameters, such as α, may be identified. In some embodiments, α may be greater than 0.2 but less than 0.3. In some embodiments, a may be greater than 0.25 but less than 0.3. In some embodiments, α may be equal to about 0.286.

Using the optimized hyperparameters and the loss function, the neural network may be trained using the expanded training set created above. The neural network may be trained and validated using optimized one or more hyperparameters described above, such as a number of hidden units, number of epochs, learning rate and dropout rate. One or more parameters may be modified during training to minimize the loss function of the neural network. In some embodiments, a leave-one-out cross validation (LOOCV) may be employed to assess the performance of the neural network during training.

The process 101 may move to step 160, where the one or more processors may output one or more refined neural network models capable of predicting a bioactivity of candidate drug compounds against at least one target miRNA. The one or more processors may train one or more neural networks to predict the bioactivity of a small molecules against at least one target miRNA. In some embodiments, the one or more processors may generate a neural network that predicts a bioactivity of an input small molecule against a plurality of target miRNAs. For example, the one or more processors may generate a neural network model that predicts the bioactivity of at least one candidate drug compound against at least the 125 known human miRNAs. In some embodiments, the one or more processors may train a plurality of neural networks, each configured to predict the bioactivity of a candidate drug compounds against a target miRNA. In some embodiments, the one or more refined neural networks may be stored on a computer readable memory.

At step 170, the one or more processors may generate a candidate drug compound by inputting a candidate drug compound with chemical data into the trained neural network model. As chemical data propagates through the neural network model, the data undergoes mathematical operations and activation functions within each layer. These operations involve weighted summations, nonlinear transformations, and activation functions that introduce nonlinearity and allow the model to capture complex relationships within the data. The output of the model, representing the predicted properties or characteristics of the candidate drug compound, is generated.

The generated output from the neural network model may be post-processed to interpret and convert it into actionable information. This could involve decoding the model's predictions, applying threshold values, or transforming the output into a suitable chemical or biological representation. Based on the predicted properties or characteristics, the processor(s) generate or select a candidate drug compound that exhibits desirable features, such as high efficacy, low toxicity, or target specificity.

To refine the generated candidate drug compound, an iterative optimization process may be employed. This process could involve modifying specific chemical features or properties of the compound while maintaining its desirable characteristics. Techniques like genetic algorithms, evolutionary strategies, or gradient-based optimization can be used to iteratively explore the chemical space and generate improved candidate compounds.

While at step 170, one or more processors may provide the generated candidate drug compound as an output, the candidate can then be further evaluated through in silico methods, such as molecular docking, molecular dynamics simulations, or ADME (absorption, distribution, metabolism, excretion) predictions, to assess its potential for further development. This evaluation helps determine the candidate compound's likelihood of exhibiting the desired biological activity, pharmacokinetic properties, and safety profile.

The process 101 may proceed to step 180 where the one or more processors may assess the candidate drug compound. The one or more processors may assess the candidate drug compound by using the one or more refined neural network models to predict the bioactivity of the candidate drug compound. As discussed below, the one or more refined neural network models may output a probability that the candidate drug compound will be bioactive against at least one target miRNA. In some embodiments, the neural network may rank a plurality of candidate drug compounds based on the predicted bioactivity of the candidate drug compounds against the at least one target miRNA. The ranking may include, for example, the top-100, top-200, top-250, top-300, top-400, top-500, top-600, top-700, top-750, top-800, top-900, top-1,000, etc. most bioactive candidate drug compounds. In some embodiments, the ranking may include one or more small molecules/candidate drug compounds known to be bioactive against the target miRNA. At step 160, the method may proceed by outputting one or more trained neural networks, and the neural networks may be capable of generating candidate drug compounds with predicted activity towards a biological target. The disclosure features multiple advantages useful for predicting novel small molecules, any of which may be used individually or in combination. For example, the disclosure may be used to create expanded data sets, then train a neural network and then the trained neural network may be used to evaluate or suggest new drug compounds. The trained neural network may be used in a variety of ways as described herein, including evaluating new drugs that may be put into the model (during or after training) or suggesting new drugs (usually after training).

Based on the predictions from the machine learning model, a chemical compound may be identified as having potential bioactivity against a specific target or disease. An optional selection process for choosing any of the chemical compounds may include factors such as the predicted potency, selectivity, safety profile, and relevance to the target or disease of interest. A chemical compound may be clinically tested after preclinical evaluation is conducted to assess the compound's safety and efficacy in vitro and in animal models. This step may involve conducting experiments to determine the compound's pharmacokinetics (absorption, distribution, metabolism, and excretion) and pharmacodynamics (mechanism of action, target engagement, and biological effects) in relevant biological systems.

In some embodiments, some or all of the preceding steps may be performed as a computer-implemented method for increasing the size of training set data for an artificial intelligence engine. As an alternative, or in combination, some of the preceding steps may be performed as a computer-implemented method for training a neural network to engine to generate candidate drug compounds affecting one or more biological targets.

In general, an example method for training a neural network to engine to generate candidate drug compounds affecting one or more biological targets, may include the steps of collecting a set of known drug compounds from a database, creating a training set comprising chemical data and biological data associated with the set of known drug compounds, wherein the chemical data includes structural information of the drug compounds and the biological data includes bioactivity information of the drug compounds towards one or more biological target, wherein each biological target has associated sequence information, calculating a sequence similarity score between biological targets based on the sequence information of the biological targets, training a neural network using the expanded training set to generate a prediction score of bioactivity of candidate drug compounds towards a biological target, wherein the contribution of each biological target towards other biological targets is weighted by the sequence similarity score, and outputting one or more refined neural network models capable of generating candidate drug compounds with predicted activity. Examples of such models have been shown to outperform comparable models by 6.18-24.67% in the top-300 (7.5%) of predictions retrieved and by 2.74-20.50% in the top-1000.

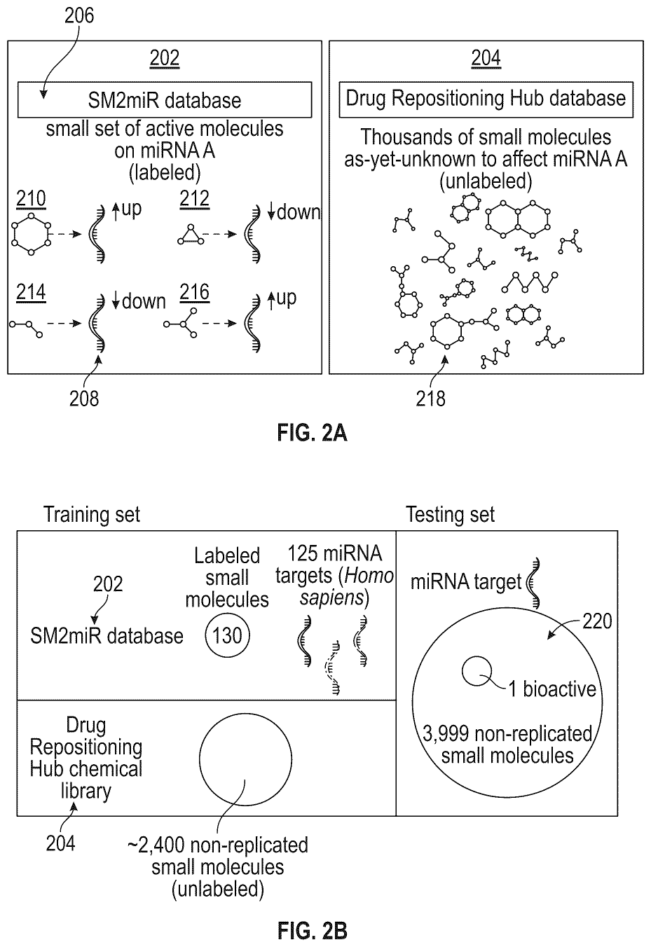

FIG. 2A depicts a first training set 202 and a second training set 204. The first training set may be stored at a first database, such as the SM2miR database 206 shown in FIG. 2A. As discussed above, the first training set may include at least one small molecule with a known bioactivity against at least one target miRNA. For example, as shown in FIG. 2A the first training set 202 includes at least four small molecules with known bioactivity against a target miRNA 208. The first small molecule 210 and the fourth small molecule 216 may increase the activity of the target miRNA 208, and the second small molecule 212 and the third small molecule 214 may decrease the activity of the target miRNA 208. In some embodiments, the first training set 202 may include chemical information and/or biological information associated with the small molecules known to have bioactivity against the at least one target miRNA 208. For example, the first training set may include a feature vector for each of the small molecules 210, 212, 214, and 216. The feature vector may include a chemical fingerprint, such as a MACCS fingerprint, and a sequence similarity score between each of the small molecules 210, 212, 214, and 216 against the target miRNA 208.

The second training set 204 may include a plurality of small molecules 218 with an unknown bioactivity towards the at least one target miRNA, such as the target miRNA 208. The second training set may include a feature vector for each small molecule of the plurality of small molecules 218. Similar to the first training set, the feature vector may include a chemical fingerprint, such as a MACCS fingerprint of the small molecule.

FIG. 2B illustrates how the first training set 202 and the second training set 204 may be combined to form an expanded training set 220. As discussed above, the expanded training set 220 may include at least one feature vector from the first training set 202 and a plurality of feature vectors from the second training set 204. For example, the expanded training set 220 may include a feature vector associated with the first small molecule 210 with known bioactivity against the target miRNA 208 and a plurality of feature vectors associated with the plurality of small molecules 218 with an unknown bioactivity toward the target miRNA 208. For example, the expanded training set may include the feature vector associated with the first small molecule 210 and 3,999 feature vectors associated with small molecules belonging to the plurality of small molecules 218 with unknown bioactivity.

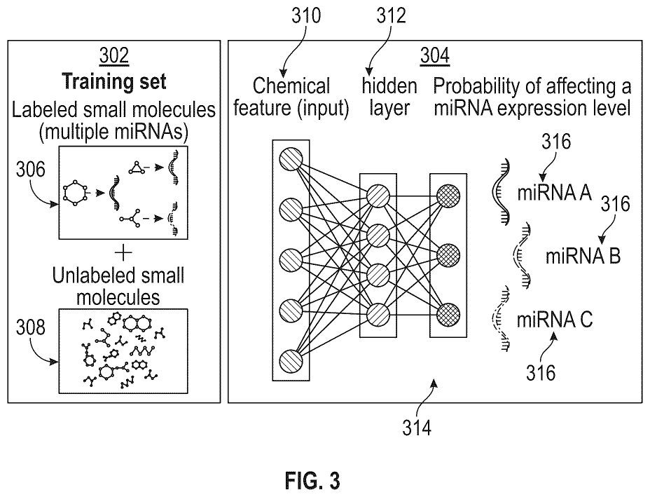

FIG. 3 illustrates an expanded training set 302 that may be used to train a neural network model 304. As discussed herein, the expanded training set 302 may include at least one feature vector from a first training set 306 that includes information about at least one small molecule with known bioactivity against a target miRNA and a plurality of feature vectors from a second training set 308 that include information about a plurality of small molecules with unknown bioactivity toward a target miRNA.

The expanded training set 302 may be used to train a neural network model 304. As discussed above, the neural network model 304 be a neural network comprised of a plurality of layers. The neural network 304 may include an input layer 310, at least one hidden layer 312, and an output layer 314. Each layer may include a plurality of nodes or neurons that may be each connected to at least one neuron in each adjacent layer. In some embodiments, the neural network may be fully connected. The input layer 310 may include binary inputs corresponding with each value in a small molecule feature vector. The at least one hidden layer 312 may include a plurality of nodes. In some embodiments, the at least one hidden layer 312 may include at least 5 nodes, at least 10 nodes, at least 15 nodes, at least 20 nodes, at least 25 nodes, at least 35 nodes, at least 40 nodes or any other suitable number of nodes. The output layer 314 may include a predicted bioactivity value for each target miRNA 316. In some embodiments, the model 304 may provide a ranking of small molecules based on the predicated bioactivity value for each small molecule.

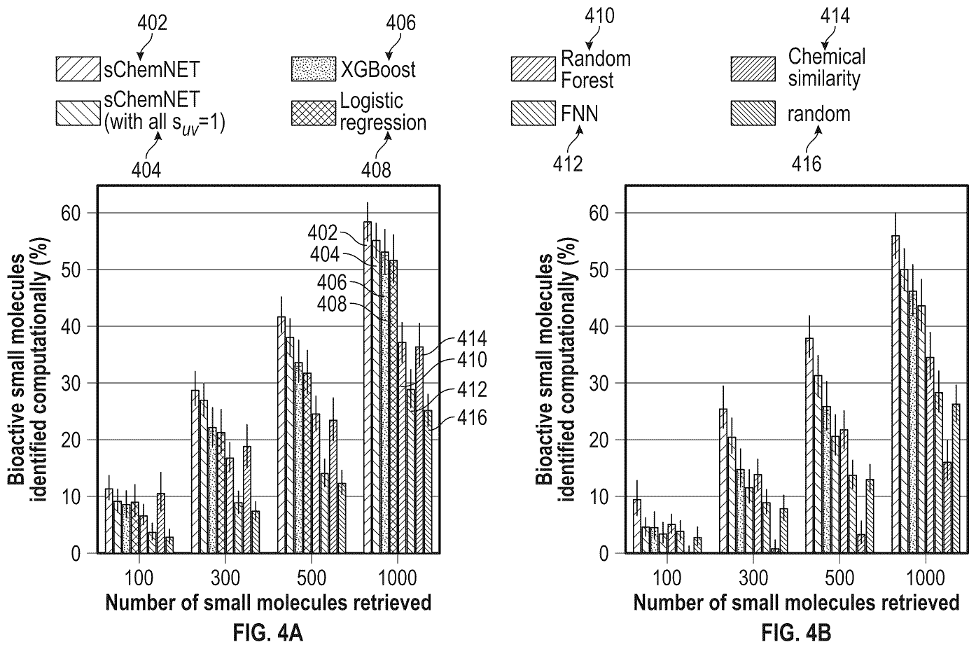

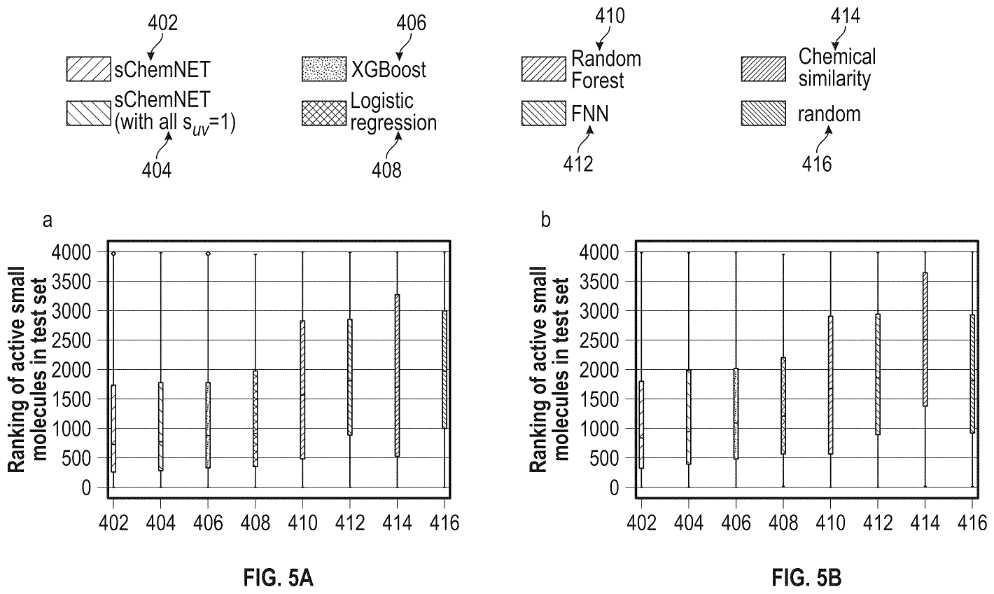

FIG. 4A, FIG. 4B, FIG. 5A, and FIG. 5B show the performance of a neural network 402 trained using methods and systems described herein compared to other machine learning models. The trained neural network may be referred to in the specification and figure as an “sChemNET” model 402. FIG. 4A, FIG. 4B, FIG. 5A, and FIG. 5B compare the performance of the sChemNET model 402 with other versions of the sChemNET Model, such as an sChemNET model where sᵤᵥ=1 404. Additionally, FIG. 4A, FIG. 4B, FIG. 5A, and FIG. 5B compare the sChemNET model 402 with existing models, including an XGBoost model 406, a logistic regression model 408, a Random Forest model 410, a Feed Forward Neural Network (“FNN”) model 412, a chemical similarity model 414, and a random model which randomly chooses small molecules 416. The chemical similarity model 414 predicts the bioactivity of small molecules using the maximum Tanimoto chemical similarity to the set of active small molecules in training set.

In FIG. 4A, FIG. 4B, FIG. 5A, and FIG. 5B, the models are shown left to right in order: the sChemNET model 402, the sChemNET_{Suv=1} model 404, the XGBoost model 406, the logistic regression model 408, the Random Forest model 410, the FNN model 412, the chemical similarity model 414, and the random model 416.

FIG. 4A shows the prediction performance of sChemNET 402 at retrieving the small molecules that alter the activity of 125 miRNA targets in Homo sapiens. sChemNET 402 outperforms the baseline methods at different numbers of small molecules retrieved by 1-9% for the top-100 of small molecules retrieved from the test set; 7-21% (top-300); 5-33% (top-500) and 8-29% in the top-1000. sChemNET_{Suv=1} 404 also achieves good prediction performance even without using sequence similarity information in the loss function but with a reduction in prediction performance of about 1.81-3.62% across the different thresholds

In examples, the following methods may be used to score small molecules in a test set: Chemical similarity baseline. Each small molecule in the test set may be scored based on the maximum chemical similarity to an active small molecule in the training set. Chemical similarities were computed using the 2D Tanimoto chemical similarity based on the binary fingerprints. Random baseline. Each small molecule in the test set may be assigned a random score sampled from a uniform distribution between 0 and 1. Machine Learning baselines. Machine learning baselines may be implemented using sklearn that work with the same dataset as sChemNET. These models include Logistic Regression, Random Forest (best hyperparameter set, ‘n_estimators’: 2, ‘min_samples_split’:10, ‘min_sample_leaf’: 3, ‘max_features’:2, ‘max_depth’: 50, ‘bootstrap’: True), XGBoost (best hyperparameter set, ‘subsample’: 0.5, ‘n_estimators’: 1000, ‘min_samples_split’: 5, ‘min_samples_leaf’: 5, ‘max_depth’: 3, ‘learning_rate’: 0.02).

FIG. 4B shows the prediction performance of sChemNET 402 for bioactive small molecules in the test set that were chemically dissimilar from those available in the training set (Tanimoto similarity <0.6). sChemNET 402 significantly outperforms the baseline methods by 5-9% in identifying the top-100 of small molecules retrieved from the test set; 10-24% (top-300); 10-40% (top-500) and; 12-34% in the top-1000. These findings suggest that sChemNET 402 would be useful in the discovery of novel small molecules against miRNA targets.

FIGS. 5A and 5B show an average predicted ranking for a known bioactive small molecule when ranking the probability scores of the 4,000 small molecules in the test set. In FIG. 5A, the analysis was performed without accounting for chemically similar compounds between training and testing sets. Conversely in FIG. 5B, the analysis included took the chemical similarity between compounds in the training set and the testing set into account. In FIG. 5B, only instances where the active small molecule in the testing set was chemically dissimilar (Tanimoto chemical similarity lower than 0.6) to active small molecules present in training set were analyzed.

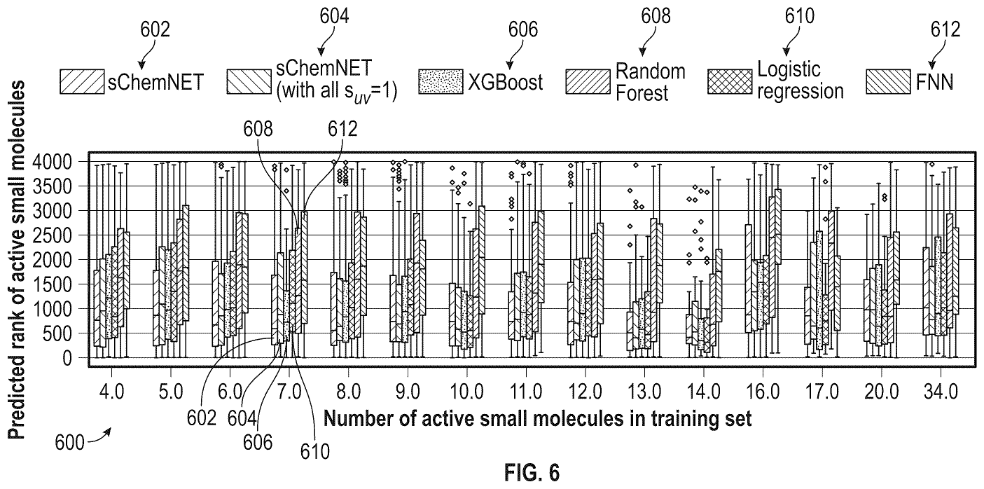

Similar to FIGS. 5A and 5B, FIG. 6 shows a plot 600 illustrating an average predicted ranking of known bioactive small molecules by sChemNET 602, sChemNET_{Suv=1} 604 and other models. The other existing models include an XGBoost model 606, a Random Forest model 608, a Logistic Regression model 610, and a Feed Forward Neural Network (“FNN”) model 612. Average rankings of known bioactive small molecules for each model are shown based on the number of known bioactive small molecules in the training set. The average rankings for each model are shown as box-and-whisker plots.

Left-to-right on the x-axis, the plot 600 shows the average ranking for known bioactive small molecules where the training set includes 4 known bioactive small molecules, 5 known bioactive small molecules, 6 known bioactive small molecules, 7 known bioactive small molecules, 8 known bioactive small molecules, 9 known bioactive small molecules, 10 known bioactive small molecules, 11 known bioactive small molecules, 12 known bioactive small molecules, 13 known bioactive small molecules, 14 known bioactive small molecules, 16 known bioactive small molecules, 17 known bioactive small molecules, 20 known bioactive small molecules, and 34 known bioactive small molecules. For each grouping, the models shown left to right are: the sChemNET model 602, the sChemNET_{Suv=1} model 604, the XGBoost model 606, the Random Forest model 608, the Logistic Regression model 610, and the FNN model 612.

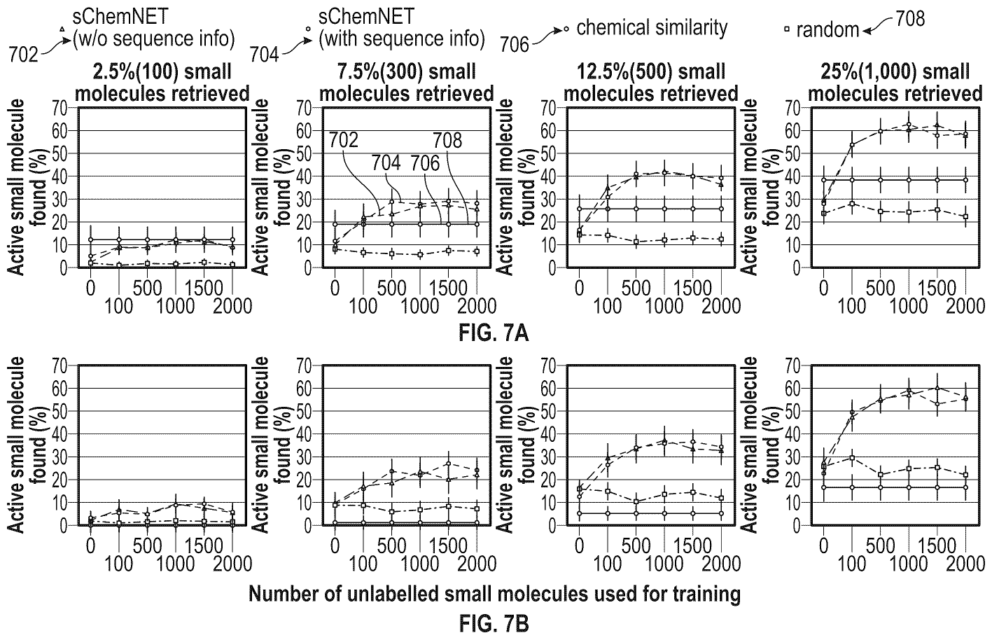

FIGS. 7A and 7B illustrate the prediction performance at retrieving active small molecules against fifty randomly selected miRNA targets in Homo sapiens from a pool of 4,000 small molecules in a leave-one-out procedure. FIGS. 7A and 7B show the percentage of known bioactive small molecules found by the sChemNET model 702, the sChemNET_{Suv=1} model 704, a chemical similarity model 706, and a random model 708. As described above, the chemical similarity model 706 predicts the bioactivity of small molecules using the maximum Tanimoto chemical similarity to the set of active small molecules in training set. The random model 708 randomly assigns a predicted bioactivity of 0 or 1 to the small molecules in the testing set.

FIGS. 7A and 7B show the percentage of known bioactive small molecules found by each model in the top 100 small molecules ranked by each model, the top 300 small molecules ranked by each model, the top 500 small molecules ranked by each model, and the top 1,000 small molecules ranked by each model. The figure shows the percentage of known active small molecules retrieved as a function of the number of unlabelled small molecules used for training the model. Similar to the Figures above, FIG. 7A shows an analysis without controlling for chemical similarity in training and testing, and FIG. 7B shows the rankings for only chemically dissimilar small molecules (2D Tanimoto chemical similarity <0.6). These figures show the real applicability of the disclosed approach for predicting novel bioactive small molecules chemically dissimilar from those available for training the model.

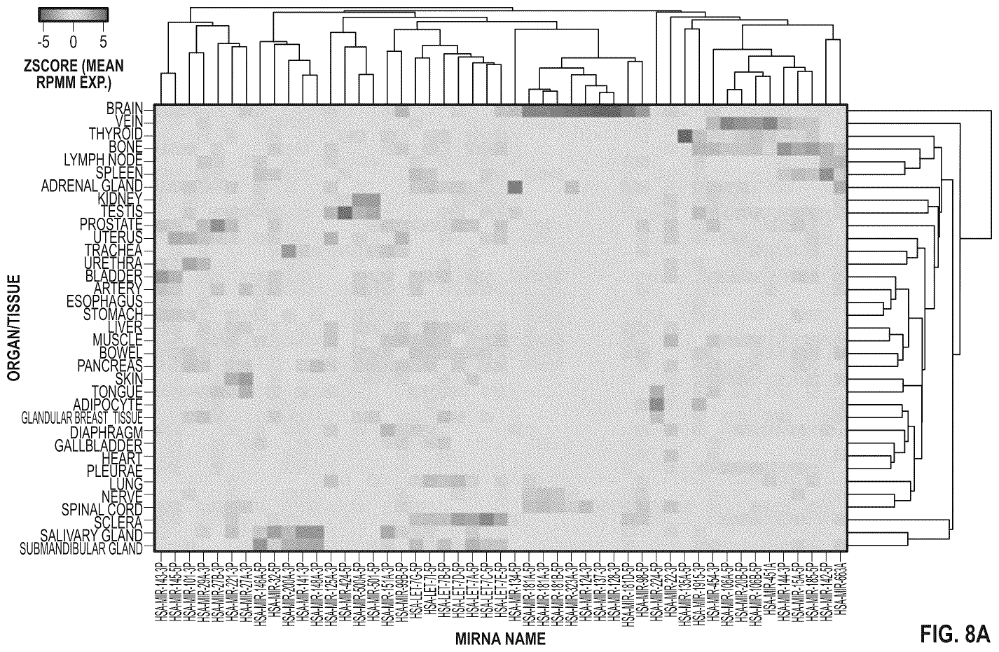

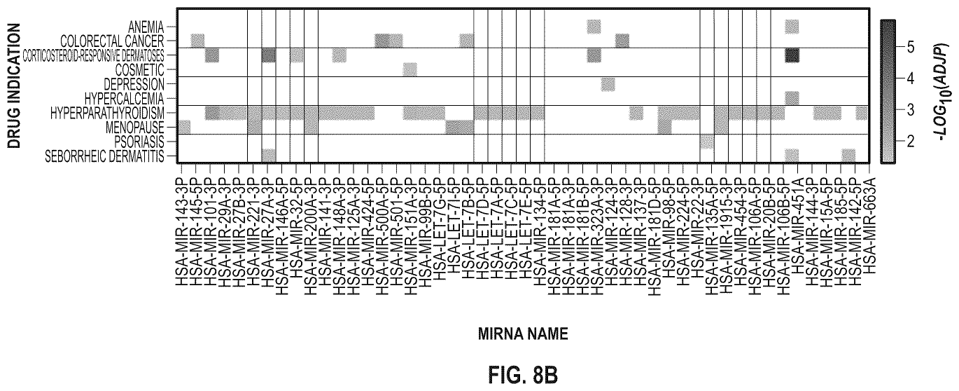

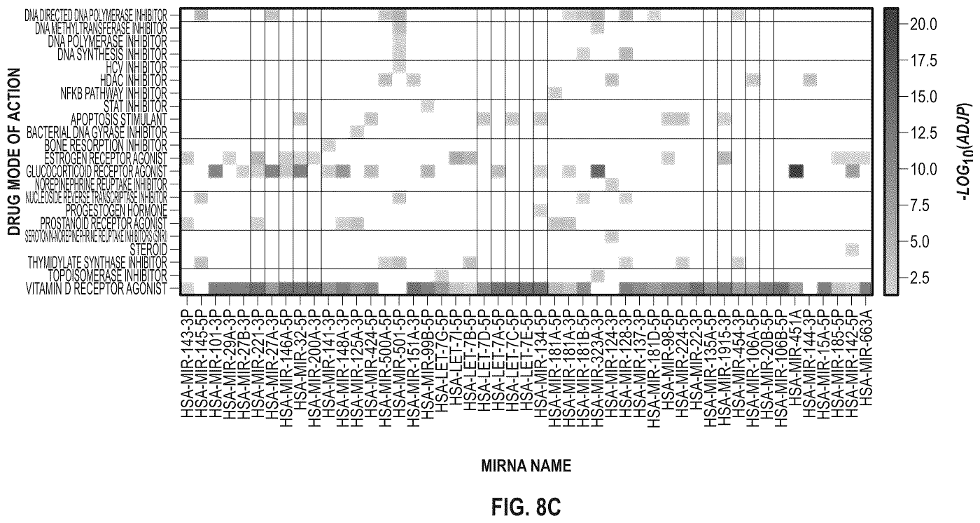

FIGS. 8A, 8B, and 8C are heatmaps showing an enriched Mode of Action (MoA) and indications of small molecules predicted by a neural network trained using the methods and systems described herein (sChemNET) to target miRNAs in Homo sapiens. In FIG. 8A, miRNAs were clustered using hierarchical clustering based on their expression levels on human tissues using the Tissue Atlas dataset, with darker colors representing higher expression. In FIG. 8B, enriched indications of predicted small molecules are shown. In FIG. 8C, enriched MoA of predicted small molecules are shown. The color is proportional to
−log₁₀ p, where p is the Benjamini-Hochberg corrected p-value

The Benjamini-Hochberg corrected p-value may be calculated using Fisher's Exact Test to keep an overall significance below 0.05. White areas indicate non-significant correlations. These figures show effectiveness of sChemNET at computationally predicting small molecules bioactive against miRNAs and further used to map specific miRNAs to the pharmacological and clinical space of drugs, that is, the space drug mode of action (MoA) and indications, respectively. Accordingly, additional biological targets and insights about possible mechanisms and follow up in vivo experimentation may be provided.

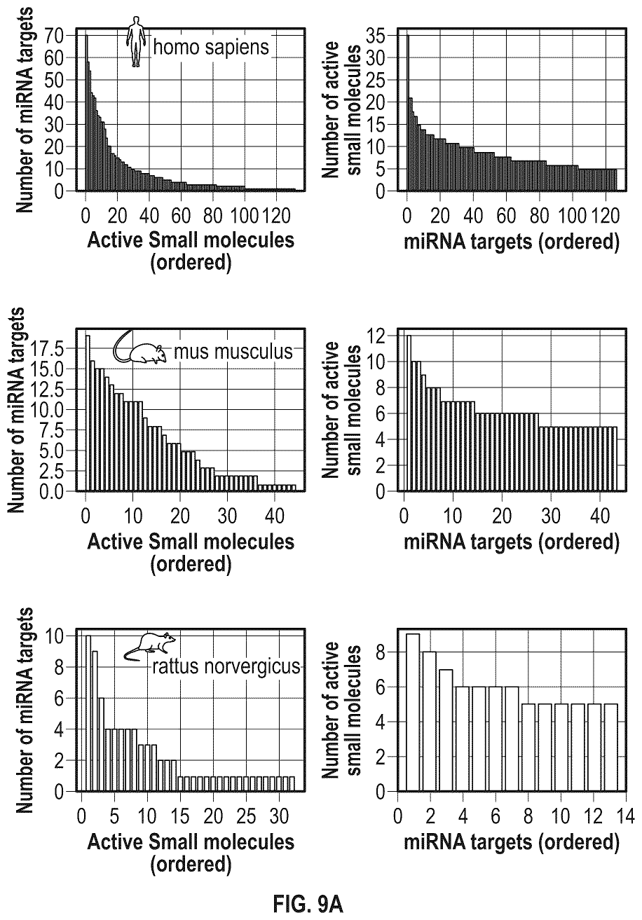

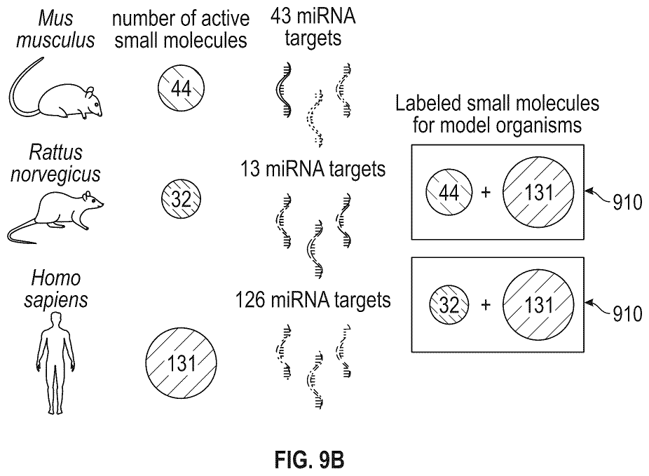

FIGS. 9A and 9B illustrate an example of a trained neural network for predicting small molecules that bind to target miRNAs in other species, such as Mus musculus and Rattus norvegicus. Although little is known about miRNA targets in humans and the small molecules that bind to them, there is even less information regarding miRNA targets in other species such as Mus musculus and Rattus norvegicus. FIG. 9A shows how in Mus musculus, only 44 bioactive small molecules and 43 target miRNAs have been identified. Similarly, only 32 small bioactive small molecules and 13 target miRNAs have been identified in Rattus norvegicus.

The systems and methods described herein may be used to train a model to predict the bioactivity of small molecules against target miRNAs in other species such as Mus musculus and Rattus norvegicus. Similar to the process described above, a first training set 910 of known bioactive small molecules may be collected or retrieved. The first training set 910 may include at least one feature vector associated with a small molecule with known bioactivity against a target miRNA. In some embodiments, the first training set may include at least one feature vector associated with a small molecule with known bioactivity against a first target miRNA in a first species and at least one feature vector associated with a small molecule with known bioactivity against a second target miRNA in a second species.

For example, as shown in FIG. 9B, the first training set 910 may include at least one feature vector associated with a small molecule known to be bioactive against at least one human target miRNA and at least one feature vector associated with a small molecule known to be bioactive against at least one mouse or rat target miRNA. In some embodiments, the first training set may include at least one feature vector associated with a known bioactive small molecule for each target miRNA in a first species and each target miRNA in a second species. Returning to the example, the first training set 910 may include at least one feature vector of a bioactive small molecule for each of the 131 human target miRNAs and at least one feature vector of a bioactive small molecule for each of the mouse or rat target miRNAs.

The first training set may be combined with a second training set to form an expanded training set, as described above. The second training set may include a plurality of feature vectors associated with a plurality of small molecules with unknown bioactivity towards the target miRNAs in another species. The expanded training set may be used to train a neural network model that predicts the bioactivity of small molecules against the target miRNAs in the other species.

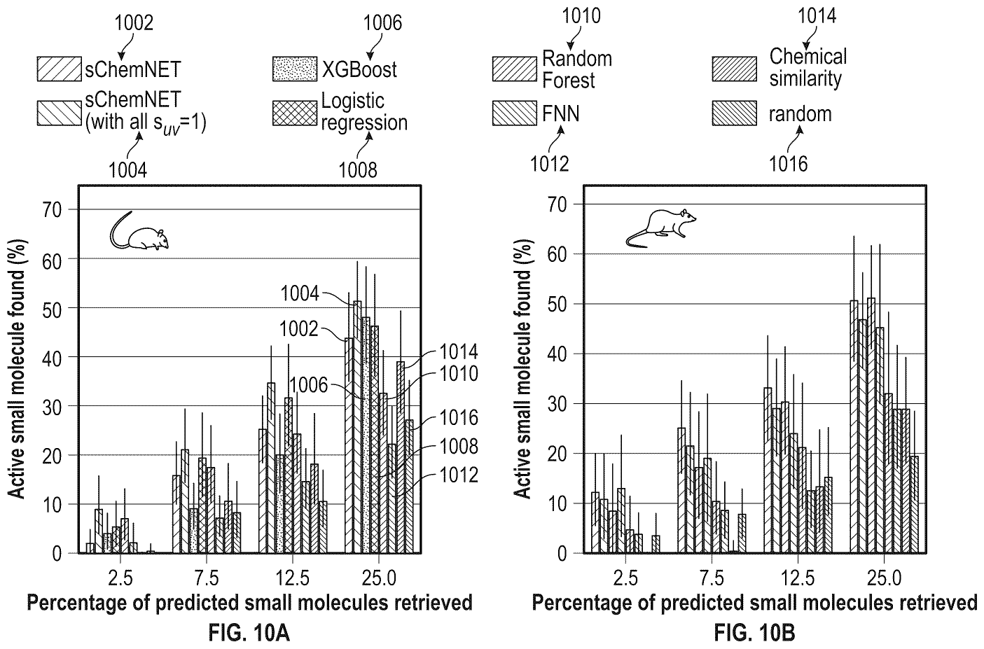

FIGS. 10A and 10B illustrate the performance of a neural network model 1002 (sChemNET) trained to predict the bioactivity of small molecules against target miRNAs in different species. FIG. 10A illustrates the performance of the model 1002 in predicting the bioactivity of small molecules in Mus musculus (mice), and FIG. 10B illustrates the performance of the model 1002 in predicting the bioactivity of small molecules in Rattus norvegicus (rats). In some embodiments, a neural network may perform best without using sequence similarity information. Here, the mouse model shows that the neural network is able to retrieve, on average, more than 43% of bioactive small molecules within the top 25% of prediction retrieved.

The neural network model 1002 may be trained using the expanded training sets described above in conjunction with FIGS. 9A and 9B. Similar to FIGS. 4A and 4B, the performance of the sChemNET model at predicting bioactivity of small molecules in mice and rats may be compared to a modified sChemNET model, sChemNET_{Suv=1} 1004, where su, in the sChemNET model is set to 1, and other existing models. The existing models included: an XGBoost model 1006, a logistic regression model 1008, a Random Forest model 1010, a Feed Forward Neural Network (“FNN”) model 1012, a chemical similarity model 1014, and a random model 1016. The chemical similarity model 1014 predicts the bioactivity of small molecules using the maximum Tanimoto chemical similarity to the set of active small molecules in training set. The random model randomly assigns a bioactivity of 0 or 1 to the small molecules. Similar to the Figures above, the models are grouped in each Figure left to right as: the sChemNET model 1002, the sChemNET_{Suv=1} model 1004, the XGBoost model 1006, the logistic regression model 1008, the Random Forest model 1010, the FNN model 1012, the chemical similarity model 1014, and the random model 1016.

FIG. 10A illustrates the percentage of small molecules known to be bioactive against target miRNAs in Mus musculus that were chemically dissimilar (Tanimoto chemical dissimilarity <0.6) and ranked by each model in the top 2.5%, top 7.5%, top 12.5%, and top 25% most bioactive small molecules. It may be observed that the sChemNET model 1002 performs best without using sequence similarity information and it is able to retrieve, on average, more than 43% of active small molecules within the top 25% of prediction retrieved.

The sChemNET model 1002 significantly outperforms the baselines and can recover more than 50% of the active small molecules within the top 25% of predictions retrieved. It may be also observed that when considering all the instances in our evaluation, chemical similarity has a good prediction performance, possibly indicating a bias towards chemically similar compounds in the small chemical dataset available for Mus musculus.

Similarly, FIG. 10B illustrates the percentage of small molecules known to be bioactive against target miRNAs in Rattus norvegicus that were chemically dissimilar (Tanimoto chemical dissimilarity <0.6) and ranked by each model in the top-100, top-300, top-500, and top-1,000 most bioactive small molecules. Similarly, FIG. 10B shows the prediction performance of the different methods for chemically dissimilar instances of active small molecules against miRNA targets in Rattus norvergicus. In this dataset, the sChemNET model 1002 outperforms the compared models by 6.18-24.67% in the top-300 (7.5%) of predictions retrieved and by 2.74-20.50% in the top-1000 (12.5%). Logistic regression performs 0.726% better than sChemNET in the top-100 (2.5%), however, this result does not hold and reverses when more small molecules are retrieved. At 25% of the molecules retrieved the neural network is able to retrieve over 50% of the known molecules in the training set.

### Expanding the Size of the Training Set

In examples, a computer-implemented method for increasing the size of training set data for an artificial intelligence engine is described. In one embodiment, the method may include providing a first training set comprising a first plurality of training samples, wherein each training sample includes associated input data labels and corresponding output labels. The method may also include providing a second training set comprising a second plurality of partially-labeled samples, wherein the partially labeled samples comprise associated input data labels without corresponding data output labels, wherein the second training set is larger than the first training set. The method may then optionally, apply a data augmentation technique to the initial training set to generate augmented training samples, wherein the data augmentation technique introduces variations in the input data while preserving the corresponding output labels.

The method may continue by combining the first training set and the second training set to form an expanded training set. Techniques for combining the datasets may depend on the size and shape of the datasets. Combining datasets of different sizes may involve merging the data in a way that preserves the information from both datasets. One approach is to use a technique called concatenation, where the smaller dataset is appended to the larger dataset. This may be done by aligning the columns or features of the two datasets and stacking the rows of the smaller dataset below the rows of the larger dataset. By using this method, the combined dataset will have a larger total sample size, incorporating the data from both sources. In general, the datasets may be useful when compatible in terms of their data types, column names, and feature representations.

When combining two datasets of different sizes into a single matrix with zero values for unlabeled entries, a common approach is to create a sparse matrix representation. The larger dataset forms the main structure of the matrix, while the smaller dataset is aligned and inserted within the appropriate rows and columns of the larger dataset. Any entries in the larger dataset that do not have corresponding data in the smaller dataset may be filled with zeros, representing the unlabeled entries.

Once the combined dataset is generated, the combined data set may be used for training a neural network using the expanded training set to generate a prediction score of bioactivity of candidate drug compounds. In order to combine the data in a meaningful way, the contribution of the second plurality of unlabeled samples in training a neural network may be scaled by a parameter α; wherein α is greater than zero.

### Improving the Performance of a Neural Network by Using a Loss Function

In examples, a computer-implemented method for training a neural network to generate candidate drug compounds affecting one or more biological targets is described. In some embodiments, the computer-implemented method may include collecting a set of known drug compounds from a database. The method may continue by creating a first training set comprising chemical data and biological data associated with the set of known drug compounds, wherein the chemical data includes structural information of the drug compounds and the biological data includes bioactivity information of the drug compounds towards one or more biological target, wherein each biological target has associated sequence information. The method may continue by creating a second training set comprising chemical data associated with drug compounds with unknown biological activity towards one or more biological targets. The method may then combine the first training set and the second training set to form an expanded training set.

The method may optionally include calculating a sequence similarity score between biological targets based on the sequence information of the biological targets. Then the method may train a neural network using the expanded training set to generate a prediction score of bioactivity of candidate drug compounds towards a biological target, wherein the contribution of each biological target towards other biological targets is weighted by the sequence similarity score, wherein the contribution of the second plurality of unlabeled samples is reduced as compared to the first plurality of labeled data in training the neural network. For example, a loss function similar to loss function of equation 2 may be used. Finally, the method may output one or more refined neural network models capable of generating candidate drug compounds with predicted activity.

### Integrating Sequence Similarity Information

In examples, a computer-implemented method for training a neural network to engine to generate candidate drug compounds affecting one or more biological targets is described. The method may include collecting a set of known drug compounds from a database, and then creating a training set comprising chemical data and biological data associated with the set of known drug compounds, wherein the chemical data includes structural information of the drug compounds and the biological data includes bioactivity information of the drug compounds towards one or more biological target, wherein each biological target has associated sequence information. The method may calculate a sequence similarity score between biological targets based on the sequence information of the biological targets. The method may then train a neural network using the expanded training set to generate a prediction score of bioactivity of candidate drug compounds towards a biological target, wherein the contribution of each biological target towards other biological targets is weighted by the sequence similarity score. Finally, the method may output one or more refined neural network models capable of generating candidate drug compounds with predicted activity.

Predicting Active and Novel Small Molecules Against miRNA Targets in Model Organisms by Cross-Species Data Integration

In examples, a computer-implemented method of where the first training set further includes chemical data and biological data includes biological data from non-human animals is described. In some embodiments, the computer-implemented method may include calculating a chemical similarity score from chemical data, and then generating a candidate drug compound by inputting a candidate drug compound with a low chemical similarity score into the trained neural network model to identify novel candidate drug compounds. The computer-implemented method may or not include a weighted contribution of each biological target towards other biological targets.

In examples, a deep learning approach may predict miRNA targets targeted by small molecules. In examples, these small molecules, and/or the targets may affect miRNA function. Examples may learn non-linear relationships between chemical features of small molecules and their miRNA target. In some examples, the learning from small bioactive miRNA-chemical datasets may be performed by using information from labelled and unlabeled chemicals. Examples can be effective for predicting molecule-miRNA associations obtained from Homo sapiens and other mammalian model organisms. In some examples, this model provides new predictive understanding of the chemical principles by which small molecules are bioactive against a particular miRNA target. In some examples, this knowledge can be used as hypothesis generator for experimental design.

The disclosure provides experimental validations in zebrafish embryos and human cells that demonstrate the small molecules predicted by sChemNET can act either directly on the miRNAs, affecting their processing or expression, or modulating the expression of genes in the miRNA-target network. Either mechanism of action may be valid, as both pathways will allow the desired output, which complements miRNA activity.

One example is α-Calcidol, which does not directly affect the levels of miR-451 or its cluster partner miR-144 but boosts blood production. The reason that α-Calcidol does not affect the levels of the erythrocyte specific miR-144 is because Dicer and miR-144 are engaged in a negative feedback loop in erythrocytes. Dicer processes miR-144, but at the same time is a target of miR-144 (PMID: 32191872), effectively canceling a potential drug-induced increase in miR-144 output.

In some examples, experimental validation on Zebrafish embryos showed successful drugs predicted to modulate the activity of miR-451 or the expression of its targets. Zebrafish embryos were incubated with different drug candidates predicted by sChemNET in combination with phenyl-thiourea (PTU), a chemical known to induce anemia due to oxidative stress when miR-451 activity is impaired, but not in wild-type embryos. 48 hours after fertilization, embryos display robust blood circulation. At this stage, the accumulation of mature erythrocytes can be easily assessed in transparent embryos using O-dianisidine, a hemoglobin-specific stain. Drugs impairing miR-451 activity induce anemia, while miR-451 boosting drugs will increase erythrocyte production (blood circulation).

Ventral images of 2-day-old embryos stained with O-dianisidine reveal hemoglobinized cells (brown staining).) for wild-type embryos and those treated with docetaxel, β-elemene and α-calcidol. Blood accumulated in the ventral region (ducts of Cuvier). A lateral view of another group of embryos to reveal accumulation of the excess of blood in the tail region upon drug treatment.

### Definitions

The various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

The hardware and data processing apparatus used to implement the various illustrative logics, logical blocks, modules and circuits described in connection with the aspects disclosed herein may be implemented or performed with a general-purpose single- or multi-chip processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some implementations, particular steps and methods may be performed by circuitry that is specific to a given function.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, i.e., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a tangible, non-transitory computer-readable medium. Computer-readable media includes both computer storage media and communication media including any medium that can be enabled to transfer a computer program from one place to another. A storage media may be any available media that may be accessed by a computer.

A software module may reside in random access memory (RAM), flash memory, read only memory (ROM), electrically programmable ROM (EPROM), electrically erasable programmable ROM (EEPROM), registers, hard disk, a removable disk, a CD ROM, or any other form of storage medium known in the art. A storage medium is coupled to the processor such that the processor can read information from, and write information to, the storage medium. In the alternative, the storage medium may be integral to the processor. Disk and disc, as used herein, includes compact disc (CD), laser disc, optical disc, digital versatile disc (DVD), floppy disk and blue ray disc where disks usually reproduce data magnetically, while discs reproduce data optically with lasers. Combinations of the above should also be included within the scope of computer readable media. The processor and the storage medium may reside in an ASIC. The ASIC may reside in a user terminal. In the alternative, the processor and the storage medium may reside as discrete components in a user terminal.

Additionally, the operations of a method or algorithm may reside as one or any combination or set of codes and instructions on a machine readable medium and computer-readable medium, which may be incorporated into a computer program product.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein. Additionally, a person having ordinary skill in the art will readily appreciate, the terms “upper” and “lower” are sometimes used for ease of describing the figures, and indicate relative positions corresponding to the orientation of the figure on a properly oriented page, and may not reflect the proper orientation of a feature as implemented.

While certain embodiments of the present disclosure have been described, these embodiments have been presented by way of example only and are not intended to limit the scope of the present disclosure. Indeed, the novel methods and systems described herein may be embodied in a variety of other forms. Furthermore, various omissions, substitutions and changes in the systems and methods described herein may be made without departing from the spirit of the present disclosure. The accompanying claims and their equivalents are intended to cover such forms or modifications as may fall within the scope and spirit of the present disclosure. Accordingly, the scope of the present disclosure is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Although features may be described above as acting in certain combinations, one or more features from a claimed combination can, in some cases, be excised from the combination, and the combination may be claimed as a subcombination or variation of a subcombination.

The features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure.

For purposes of this disclosure, certain aspects, advantages, and novel features are described herein. Not necessarily all such advantages may be achieved in accordance with any particular embodiment. Thus, for example, those skilled in the art will recognize that the present disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z. Thus, as used herein, a phrase referring to “at least one of X, Y, and Z” is intended to cover: X, Y, Z, X and Y, X and Z, Y and Z, and X, Y and Z.

The headings provided herein, if any, are for convenience only and do not necessarily affect the scope or meaning of the devices and methods disclosed herein.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount.

The scope of the present disclosure is not intended to be limited by the specific disclosure of embodiments in this section or elsewhere in this specification and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

The term “Training Data” is used herein to mean the dataset used to train a machine learning model. It consists of input features and corresponding target labels or outcomes. The model learns patterns and relationships in the training data to make predictions on new, unseen data.

The term “Feature Engineering” is used herein to mean the process of selecting, transforming, and extracting relevant features from raw data to enhance the performance of a machine learning model. It involves techniques such as normalization, scaling, encoding categorical variables, dimensionality reduction, and creating new derived features.

The term “Model Architecture” is used herein to mean the structure and arrangement of layers, nodes, and connections in a machine learning model. It determines how the model processes and learns from the input data, enabling it to make predictions or decisions.

The term “Loss Function” is used herein to mean function that quantifies the discrepancy between the predicted output of a machine learning model and the actual target labels. It serves as the basis for model optimization during the training process.

The term “Hyperparameters” is used herein to mean parameters that are not learned directly from the data but are set before the training process. They control the behavior of the machine learning model, such as learning rate, regularization strength, and architecture configurations.

The term “Validation Dataset exemplary” is used herein to mean a subset of the training data that is used to assess the performance of the machine learning model during training. It provides an estimate of how well the model generalizes to new, unseen data.

The word “Overfitting exemplary” is used herein to mean a phenomenon in machine learning where a model performs extremely well on the training data but fails to generalize to new, unseen data. It occurs when the model becomes too complex or captures noise in the training data.

The term “Feature Selection” is used herein to mean the process of selecting a subset of relevant features from the available input data. It aims to reduce dimensionality, improve model interpretability, and enhance model performance by focusing on the most informative features.

The term “Cross-Validation” is used herein to mean a technique used to assess the performance and generalization capabilities of a machine learning model. It involves partitioning the dataset into multiple subsets and iteratively training and evaluating the model on different subsets to obtain robust performance estimates.

### EMBODIMENTS

The following are non-limiting examples of certain embodiments of systems and methods of characterizing coronary plaque. Other examples and embodiments may include one or more other features, or different features, that are discussed herein.

In some embodiments, a computer-implemented method for training a neural network to generate candidate drug compounds may include creating a first training set comprising structural information of drug compounds and bioactivity information of the drug compounds towards a biological target.

In examples, creating a first training set comprising structural information of drug compounds and bioactivity information of the drug compounds towards a biological target; creating a second training set comprising structural information of drug compounds with unknown biological activity towards the biological target.

In examples, combining the first training set and the second training set to form an expanded training set. In examples, training a neural network using the expanded training set.

In examples, outputting one or more refined neural network models capable of generating a prediction score of bioactivity of candidate drug compounds towards the biological target.

In examples, a computer-implemented method may include training a neural network using the expanded training set comprises scaling the relative contribution of the second training set versus the first training set by a parameter that is greater than zero and less than one.

In examples, a computer-implemented method may include generating a candidate drug compound by inputting a candidate drug compound with chemical data into the trained neural network.

In examples, a computer-implemented method may include assessing the candidate drug compound predicted activity using the trained neural network model.

In examples, a computer-implemented method may include predicting bioactive small molecules that are chemically dissimilar from those available for training the neural network model.

In examples, a computer-implemented method any chemically dissimilar small molecules may have a Tanimoto similarity of less than 0.6 compared to the small molecules available for training the neural network model.

In examples, the prediction performance may be greater than 40%, as evaluated by a leave-one-out cross-validation (LOOCV) procedure and reported as the recall of active small molecules amongst the 1000 small molecules retrieved from the test set.

In examples, the prediction performance is greater than at least one of 50%, 55%, 60%, and 65%.

In examples, a first training set is smaller than the second training set.

In examples, the first training set is ten times smaller than the second training set.

In examples, the first training set is smaller than the second training set by a factor from 100 to 5,000.

In examples, the first training set comprises fewer than ten known bioactive drug compounds.

In examples, the first training set comprises fewer than five known bioactive drug compounds.

In examples, the neural network is applied to assess candidate drug compounds to modulate at least one of miRNA, mRNA, and protein targets.

In examples, the neural network is applied to at least one of the following targets: FLT3, ALK, IGF1R, and EGFR.

In examples, the neural network is applied to at least one of the following targets: miR-10300, miR-155, miR-10b, and miR-181.

In examples, any of the disclosed examples may be implemented by computer-readable storage medium storing instructions that, when executed by one or more processors, cause the one or more processors to perform the disclosed examples.

In examples, any of the disclosed examples may be implemented by a system for training a neural network to generate candidate drug compounds, the system comprising: a memory; and a module stored on the memory configured to cause one or more processors to perform the examples.

In examples, the disclosed methods may expand the size of a training set. For example, a computer-implemented method for increasing the size of training set data for an artificial intelligence engine, may include providing a first training set comprising a first plurality of training samples, where each training sample comprises associated input data labels and corresponding output labels.

In examples, a computer-implemented method may include providing a second training set comprising a second plurality of partially-labeled samples, wherein the partially labeled samples comprise associated input data labels without corresponding data output labels. In some examples, the second training set is larger than the first training set.

In examples, a computer-implemented method may include combining the first training set and the second training set to form an expanded training set.

In examples, a computer-implemented method may include training a neural network using the expanded training set to generate a prediction score of bioactivity of candidate drug compounds. In some examples, the contribution of the second plurality of unlabeled samples in training a neural network is scaled by a parameter α; wherein α is greater than zero. In some examples, the input data labels are chemical fingerprints of a set of chemical compounds, and the data output labels are biological activity of the set of chemical compounds towards a biological target.

In some examples, the chemical fingerprint are determined as at least one of: MACCS fingerprints, Daylight fingerprints, and RDKit fingerprints.

In some examples, the set of chemical compounds comprises compounds within at least one of the SM2miR database and the Drug Repositioning Hub.

In some examples, the second training set comprises partially labeled samples that represent a wider range of chemical variety than the first training set comprising the first plurality of training samples.

In some examples, the chemical variety corresponds to the vector space of a chemical feature vector based on a chemical fingerprint.

In some examples, the second training set comprises partially labeled samples that are mapped to lower probability scores than the first training set comprising the first plurality of training samples.

In some examples, the second training set comprises partially labeled samples, wherein the average probability score of the partially labeled samples is less than the probability scores than the first training set comprising the first plurality of training samples.

In some examples, the second training set comprises partially labeled samples with associated input data labels and corresponding output labels comprises a set of non-zero latent features that encode the biological interplay between drugs and biological activity.

In some examples, the set of non-zero latent features is determined to be present by an improved prediction performance relative to a neural network trained without the expanded training set.

In examples, a computer-implemented method may include training the neural network using the expanded training set by employing a non-negative matrix decomposition model.

In examples, a computer-implemented method may include training the neural network using the expanded training set by employing zero-driven regularization in a non-negative matrix decomposition model.

In the disclosed examples, the methods may be stored on a computer-readable storage medium storing instructions that, when executed by one or more processors, cause the one or more processors to perform the disclosed methods.

In examples, any of the disclosed examples may be implemented by a system for expanding the size of the training set, the system comprising: a memory; and a module stored on the memory configured to cause one or more processors to perform any of the steps of the preceding examples.

Some examples may include improving the performance of the neural network by using a loss function including the features of the disclosure, including any combination of the features of chemical similarity, unlabeled data, expanded data sets, and sequence similarity.

A computer-implemented method for training a neural network to engine to generate candidate drug compounds affecting one or more biological targets may include collecting a set of known drug compounds from a database.

In examples, a computer-implemented method may include creating a first training set comprising chemical data and biological data associated with the set of known drug compounds.

In some examples, the chemical data comprises structural information of the drug compounds and the biological data comprises bioactivity information of the drug compounds towards one or more biological target;

In some examples, each biological target has associated sequence information;

In examples, a computer-implemented method may include creating a second training set comprising chemical data associated with drug compounds with unknown biological activity towards one or more biological targets;

In examples, a computer-implemented method may include combining the first training set and the second training set to form an expanded training set;

In examples, a computer-implemented method may include calculating a sequence similarity score between biological targets based on the sequence information of the biological targets;

In examples, a computer-implemented method may include training a neural network using the expanded training set to generate a prediction score of bioactivity of candidate drug compounds towards a biological target;

In some examples, the contribution of each biological target towards other biological targets is weighted by the sequence similarity score;

In some examples, the contribution of the second plurality of unlabeled samples is reduced as compared to the first plurality of labeled data in training the neural network; and

In some examples, a computer-implemented method may include outputting one or more refined neural network models capable of generating candidate drug compounds with predicted activity.

In examples, a computer-implemented method may include generating a candidate drug compound by inputting a drug compound with chemical data into the trained neural network model and assessing the candidate drug compound predicted activity using the trained neural network model.

In some examples, biological data for all biological targets are applied to each biological target.

In some examples, unlabeled drug compounds are assigned near zero initial prediction scores towards each biological target during the training of the neural network.

In examples, a computer-implemented method may include training a neural network using the expanded training set comprises using a Bayesian optimization approach to a hyperparameter for the contribution of the second plurality of unlabeled samples based on a leave-one-out cross validation of a drug compound known to target a biological target not included in any training sets.

In the disclosed examples, the methods may be stored on a computer-readable storage medium storing instructions that, when executed by one or more processors, cause the one or more processors to perform the disclosed methods.

In examples, any of the disclosed examples may be implemented by a system including a memory; and a module stored on the memory configured to cause one or more processors to perform any of the steps of the preceding examples.

In examples a computer-implemented method for training a neural network to engine to generate candidate drug compounds affecting one or more biological targets, the computer-implemented method may include collecting a set of known drug compounds from a database.

In examples, a computer-implemented method may include creating a training set comprising chemical data and biological data associated with the set of known drug compounds.

In examples, a computer-implemented method may include wherein the chemical data comprises structural information of the drug compounds and the biological data comprises bioactivity information of the drug compounds towards one or more biological target.

In some examples, each biological target has associated sequence information.

In examples, a computer-implemented method may include calculating a sequence similarity score between biological targets based on the sequence information of the biological targets.

In examples, a computer-implemented method may include training a neural network using the expanded training set to generate a prediction score of bioactivity of candidate drug compounds towards a biological target.

In some examples, the contribution of each biological target towards other biological targets is weighted by the sequence similarity score.

In examples, a computer-implemented method may include outputting one or more refined neural network models capable of generating candidate drug compounds with predicted activity.

In the disclosed examples, the methods may be stored on a computer-readable storage medium storing instructions that, when executed by one or more processors, cause the one or more processors to perform the disclosed methods.

In examples, any of the disclosed examples may be implemented by a system for expanding the size of the training set, the system comprising: a memory; and a module stored on the memory configured to cause one or more processors to perform any of the steps of the preceding examples. Further examples of embodiments are listed below in Claim Sets #1-#4.

What is claimed is:

1. A computer-implemented method for training a neural network to generate candidate drug compounds, the method comprising:
creating a first training set comprising structural information of drug compounds and bioactivity information of the drug compounds towards a biological target;
creating a second training set comprising structural information of drug compounds with unknown biological activity towards the biological target;
combining the first training set and the second training set to form an expanded training set; and
training a neural network using the expanded training set,
outputting one or more refined neural network models capable of generating a prediction score of bioactivity of candidate drug compounds towards the biological target.

2. The computer-implemented method of claim 1, wherein training a neural network using the expanded training set comprises scaling the relative contribution of the second training set versus the first training set by a parameter that is greater than zero and less than one.

3. The computer-implemented method of claim 1, further comprising generating a candidate drug compound by inputting a candidate drug compound with chemical data into the trained neural network.

4. The computer-implemented method of claim 1, further comprising assessing the candidate drug compound predicted activity using the trained neural network model.

5. The computer-implemented method of claim 1, further comprising predicting bioactive small molecules that are chemically dissimilar from those available for training the neural network model.

6. The computer-implemented method of claim 5, wherein the chemically dissimilar small molecules have a Tanimoto similarity of less than 0.6 compared to the small molecules available for training the neural network model.

7. The computer-implemented method of claim 2, wherein the prediction performance is greater than 40%, as evaluated by a leave-one-out cross-validation (LOOCV) procedure and reported as the recall of active small molecules amongst the 1000 small molecules retrieved from the test set.

8. The computer-implemented method of claim 7, wherein the prediction performance is greater than at least one of 50%, 55%, 60%, and 65%.

9. The computer-implemented method of claim 1, wherein the first training set is smaller than the second training set.

10. The computer-implemented method of claim 9, wherein the first training set is ten times smaller than the second training set.

11. The computer-implemented method of claim 9, wherein the first training set is smaller than the second training set by a factor from 100 to 5,000.

12. The computer-implemented method of claim 1, wherein the first training set comprises fewer than ten known bioactive drug compounds.

13. The computer-implemented method of claim 1, wherein the first training set comprises fewer than five known bioactive drug compounds.

14. The computer-implemented method of claim 1, wherein the neural network is applied to assess candidate drug compounds to modulate at least one of miRNA, mRNA, and protein targets.

15. The computer-implemented method of claim 11, wherein the neural network is applied to at least one of the following targets: FLT3, ALK, IGF1R, and EGFR.

16. The computer-implemented method of claim 11, wherein the neural network is applied to at least one of the following targets: miR-10300, miR-155, miR-10b, and miR-181.

17. A computer-readable storage medium storing instructions that, when executed by one or more processors, cause the one or more processors to perform the method of claim 1.

18. A system for training a neural network to generate candidate drug compounds, the system comprising:
a memory; and
a module stored on the memory configured to cause one or more processors to perform the method of claim 1.

19. A computer-implemented method for training a neural network to engine to generate candidate drug compounds affecting one or more biological targets, the method comprising:
collecting a set of known drug compounds from a database;
creating a first training set comprising chemical data and biological data associated with the set of known drug compounds;
wherein the chemical data comprises structural information of the drug compounds and the biological data comprises bioactivity information of the drug compounds towards one or more biological target;
wherein each biological target has associated sequence information;
creating a second training set comprising chemical data associated with drug compounds with unknown biological activity towards one or more biological targets;
combining the first training set and the second training set to form an expanded training set;
calculating a sequence similarity score between biological targets based on the sequence information of the biological targets;
training a neural network using the expanded training set to generate a prediction score of bioactivity of candidate drug compounds towards a biological target;
wherein the contribution of each biological target towards other biological targets is weighted by the sequence similarity score;
wherein the contribution of the second plurality of unlabeled samples is reduced as compared to the first plurality of labeled data in training the neural network; and
outputting one or more refined neural network models capable of generating candidate drug compounds with predicted activity.

20. The computer-implemented method of claim 19, further comprising generating a candidate drug compound by inputting a drug compound with chemical data into the trained neural network model and assessing the candidate drug compound predicted activity using the trained neural network model.

21. The computer-implemented method of claim 19, wherein biological data for all biological targets are applied to each biological target.

22. The computer-implemented method of claim 19, wherein unlabeled drug compounds are assigned near zero initial prediction scores towards each biological target during the training of the neural network.

23. The computer-implemented method of claim 19, wherein training a neural network using the expanded training set comprises using a Bayesian optimization approach to a hyperparameter for the contribution of the second plurality of unlabeled samples based on a leave-one-out cross validation of a drug compound known to target a biological target not included in any training sets.

24. A computer-readable storage medium storing instructions that, when executed by one or more processors, cause the one or more processors to perform the method of claim 19.

25. A system for training a neural network to generate candidate drug compounds, the system comprising:
a memory; and
a module stored on the memory configured to cause one or more processors to perform the method of claim 19.
